(19) United States
(12) Patent Application Publication
(10) Pub. No.: US 2009/0088721 A1
(43) Pub. Date: Apr. 2, 2009

(54) Enhanced retinal delivery of a nucleic acid through iontophoresis

(75) Inventors: Therese de Bizemont, Paris (FR); Florian Sennlaub, Paris (FR); Francine Behar-Cohen, Paris (FR); Yves Courtois, Boulogne (FR); Andrieu Charlotte, Rotterdam (NL)

Correspondence Address: FOLEY & LARDNER LLP, 111 HUNTINGTON AVENUE, 26TH FLOOR, BOSTON, MA, 02199-7610, US

(73) Assignee: EYEGATE PHARMA S.A.S.

(21) Appl. No.: 11/999,638

(22) Filed: Dec. 5, 2007

Related U.S. Application Data
(63) Continuation-in-part of application No. 10/965,905, filed on Oct. 18, 2004.
Application No. 10/965,905 is a continuation of application No. 09/836,439, filed on Apr. 17, 2001, now abandoned.
(60) Provisional application No. 60/873,233, filed on Dec. 5, 2006.

(51) Int. Cl. A61K 31/7088; A61N 1/30

(52) U.S. Cl. 604/501; 514/44

(57) ABSTRACT

The present invention provides a device and a method for the enhanced retinal delivery of nucleic acid therapeutics utilizing iontophoresis to evoke a transient elongation of the Müller Cells of a mammalian eye. The enhanced retinal deposition can be achieved by either a topical application, subconjunctival, or an intravitreal injection of the nucleic acid composition followed by, preceded by, or administered simultaneously with the iontophoretic application. The present invention thus provides a particularly advantageous method for the treatment of ocular diseases comprising the in vivo administration of a nucleic acid capable of alleviating the symptoms of a disease, the delivery of the nucleic acid being enhanced by using iontophoresis. This method can be applied particularly to the diseases of the retina resulting from an alteration of a gene expression and/or the over-expression of particular growth factors. The diseases include, but are not limited to, human ocular retinopathies including, neovascular diseases (Age-Related Macular Edema, Diabetic Retinopathies, Diabetic Macular Edema, etc.) and inherited retinopathies such as retinitis pigmentosa.

### FIELD OF THE INVENTION

This invention relates to devices and methods for the enhanced delivery of a nucleic acid to tissues of the eye. More specifically, the methods of the present invention utilize iontophoresis to evoke a transient elongation of the Müller Cells of a mammalian eye to enhanced retinal delivery of a nucleic acid therapeutic or diagnostic agent.

### BACKGROUND OF THE INVENTION

Methods are known for introducing drug, such as nucleic acid, into target cells or tissues such as by topically applying to or injection into tissue, the use of techniques such as electroporation, iontophoresis, the encapsulation of nucleic acid in colloidal systems, such as liposomes or polymeric spheres or other chemical carriers or the use of a viral or non-viral vector.

While the use of conventional delivery systems has been widely investigated, there still exist many problems that are often associated with the in vivo introduction of nucleic acid into eukaryotic cells. Typically only a small percentage of cells targeted for transfection with a heterologous nucleic acid actually express the gene of interest at satisfying levels, notably the protein of interest. In addition, some therapeutic compositions, such as those that include synthetic oligonucleotides, are very expensive, toxic and degradable, consequently, requiring localized application and efficient internalization into the target cells.

Among the methods directed to enhancing the in vivo transfer of nucleic acid into target cells, electroporation can be particularly cited. Electroporation is a means of increasing the permeability of a cell membrane and/or at a portion of cells of a targeted tissue, to a chemical agent such as nucleic acids, wherein the increased permeability is caused by application of high pulse voltage across the cell or at least a portion of the tissue. The increased permeability allows transport, or migration, of chemical agents through the tissue or across cell membranes into cells if the tissue or the cells are in the presence of a suitable chemical agent.

Electroporation is typically carried out by applying high voltage pulses between a pair of electrodes that are applied to the tissue surface. The voltage must be applied in proportional to the distance between the electrodes. When the space between the electrodes is too great, the generated electric field penetrates deep into the tissue where it causes unpleasant nerve and muscle reaction.

Iontophoresis is a technique that was proposed in 1747 by Verrati and consists in the administration, in particular of medicaments, into the body through the tissues using an electric field involving a small voltage. An electrode is arranged at the site to be treated while a second electrode, intended to close the electric circuit, is placed at another site on the body. The electric field facilitates the migration of the active products, and/or increases cellular permeability to the products that are preferably ionized. This is commonly used transdermal technique for treating skin or rheumatologic diseases. It has also been shown that gene induction can be achieved through the ex vivo iontophoretic delivery of oligonucleotides in an ocular rabbit model.

It is known that iontophoresis wherein low voltage is applied between widely spaced electrodes can transport charged molecules through existing pathways and/or creating pathways. However, it is also known that the volumes of molecules transported are very small, and insufficient for in vivo applications in specific tissues.

From the foregoing, it will be appreciated that it would be an advancement in the art to provide a simple and efficient method for enhancing the in vivo delivery of nucleic acid into retinal cells, particularly for ocular therapy or diagnostic purposes.

### SUMMARY OF THE INVENTION

To overcome the existing needs, materials and methods are disclosed comprising the simultaneous use of both electroporation and iontophoresis for acid nucleic delivery in the patent document U.S. Pat. No. 6,009,345, issued Dec. 28, 1999. The present invention provides devices and methods for the enhanced delivery of a nucleic acid to tissues of the eye.

In one embodiment, the methods of the present invention utilize iontophoresis to evoke a transient elongation of the Müller Cells of a mammalian eye to enhanced retinal delivery of a nucleic acid therapeutic or diagnostic agent. The method comprises transiently elongating Müller cells of a mammal retina by a step of iontophoresis; and administering a composition comprising the nucleic acid to the mammalian eye, wherein the step of iontophoresis transiently elongates the Müller cells of the mammal retina and enhances the in vivo delivery of the nucleic acid into the retinal cells of the mammalian eye. The step of iontophoresis can be carried out prior to, during, or after the step of administering the nucleic acid composition.

The nucleic acid can be either a therapeutic agent or a diagnostic agent. The nucleic acid can be a deoxyribonucleic acid (“DNA”), a ribonucleic acid (“RNA”), and a chimeric nucleic acid comprising both DNA and RNA bases, including but not limited to, an oligonucleotide DNA, an anti-sense DNA, a plasmid DNA, a component of a plasmid DNA, a vector, an expression cassette, a chimeric DNA sequence, a chromosomal DNA, a stabilized DNA, an aptamer, a stabilized aptamer, an oligonucleotide RNA, a transfer RNA (tRNA), a short interfering RNA (siRNA), a small nuclear RNA (snRNA), a ribosomal RNA (rRNA), an mRNA (messenger RNA), a micro RNA (miRNA), a short hair-pin RNA (shRNA), an antisense RNA, a ribozyme, a stabilized RNA sequence, a chimeric RNA sequence, a chimeric DNA/RNA oligonucleotide, an aptameric oligonucleotide, or a derivative of any of these nucleic acid. In one embodiment, the nucleic acid is an oligonucleotide DNA or an oligonucleotide RNA, optionally with phosphorothioates linkages. In an alternative embodiment, the nucleic acid is a single-stranded nucleic acid, a double-stranded nucleic acid, a triple-stranded nucleic acid, or a quadruple-stranded nucleic acid. In yet another embodiment, the nucleic acid is in a linear or circular form. In yet another embodiment, the nucleic acid is a single stranded oligonucleotide DNA (ssODN) or a single stranded oligonucleotide RNA (ssORN).

The nucleic acid composition can be administered by a topical instillation on the eye, by a topical instillation on the eyelid, or by an injection into the mammalian eye (should we include by direct ocular iontophoresis?). The topical instillation can be administered in the form of a liquid solution, a paste, of a hydrogel. The topical instillation can be embedded in a foam matrix or supported in a reservoir. The injection into the mammalian eye can be an intracameral injection, an intracorneal injection, a subconjonctival injection, a subtenon injection, a subretinal injection, an intravitreal injection, and an injection into the anterior chamber.

The step of iontophoresis can be an ocular or a transpalpebral iontophoresis. In one embodiment, the transpalpebral iontophoresis is an anionic or a cationic iontophoresis performed with a current of about 1-5 mA for about 1-7 minutes. In an alternative embodiment, the transpalpebral iontophoresis is an anionic or a cationic iontophoresis performed with a current of about 1-3 mA for about 3-6 minutes. In yet another embodiment, the transpalpebral iontophoresis is a cationic iontophoresis performed with a current of about 2 mA for up to 5 minutes.

### BRIEF DESCRIPTION OF THE DRAWINGS

FIGS. 1A-C provide photomicrographs of histological sections of rat retina stained with hemalun. FIG. 1A: Control retina. FIG. 1B: Retina after injection into the vitreous of the biotynilated chimeroplast. No staining is observed in the retina or in the RPE showing that no chimeroplast has penetrated into the retina. FIG. 1C: Retina after injection into the vitreous of the chimeroplast, followed by the iontophoresis of saline. There is a clear brown DAB staining in the retinal layers, in the RPE and in the choroid, showing that the penetration of the chimeroplast has been enhanced by the application of the current.

FIG. 2 provides photomicrographs of a restriction fragment length analysis of β-cGMP phosphodiesterase cDNA. RT-PCR were performed with rd β-PDE mRNA specific primers on extracted retinae at postnatal day 27 (except for lanes 4-7 analyzed at postnatal day 10). The rd nonsense point mutation in codon 347 creates a DdeI restriction site and removes a BsaAI site from the wild-type sequence. Digesting the 359 bp β-PDE cDNA with BsaAI or DdeI yields two diagnostic fragments of 120 bp and 239 bp. This method allows the differentiation of the mutated sequence rd/rd (DdeI sensitive) from the wild-type one +/+ (BsaAI sensitive) at the mRNA level. The gel in FIG. 2 represents the restriction fragment length analysis by electrophoresis separation: lanes 1-3: for the wild-type cCDA sequence (+/+) without treatment; and lanes 4-18: for the mutated sequence (rd/rd) without treatment (lanes 4-6), with water injection treatment (lanes 7-9), with chimeroplast injection without iontophoresis transfer (lanes 10-12), with chimeroplast injection with iontophoresis transfer (lanes 13-15), with control chimeroplast injection with iontophoresis transfer (lanes 16-18).

FIGS. 3A and 3B provide photomicrographs illustrating rod survival by immunostaining. FIG. 3A: The amount of rod-photoreceptors was counted on flat-mounted retina of chimeraplast treated animals (“active chimera”) and control (“scrambled chimera”) at postnatal day 27 (P27). Results are expressed as mean±standard error of the mean (SEM). FIG. 3B: Opsin-immunohistochemistry has been performed on whole-mounted retina. Scanned photograph by fluorescence microscopy of flat-mounted retina of chimeraplast treated animals (“active chimera”, right picture) and control (“scrambled chimera”, left picture).

FIG. 4 provides photomicrographs illustrating penetration of ODNs to retinal cells at one hour after injection of labeled ODNs with or without prior saline iontophoresis. PN7 rd1/rd1 eye section after intravitreal injection of labeled ODNs without prior saline iontophoresis (Panel A). PN7 rd1/rd1 eye section after intravitreal injection of Hex without prior saline iontophoresis (Panel B). Iontophoresis device is composed by an eye-glass-shaped electrode made with aluminum foil and single-use disposable medical grade hydrophilic polyurethane sponge and a return electrode connected to the neck of the mouse (arrow) (Panel C). PN7 rd1/rd1 eye section after intravitreal injection of labeled ODNs with prior cathodal saline iontophoresis (Panel D). PN7 rd1/rd1 eye section after intravitreal injection of Hex with prior cathodal saline iontophoresis (Panel E). PN7 rd1/rd1 eye section after intravitreal injection of PBS with prior cathodal saline iontophoresis (Panel F). Higher magnifications showing retina structures from eyes (A), (D) and (F) are shown in (a), (d) and (f), respectively. Insets show high magnification of the correspondent picture. ONL: outer nuclear layer, INL: inner nuclear layer, GCL: ganglion cell layer. Scale bars: A, D, and F (×2.5), 1 mm; a, B, d, E and f (×25), 100 μm; insets, 10 μm.

FIG. 5 depicts the analysis of varied iontophoresis conditions on the ODNs delivery to the ONL. Relative fluorescent intensities in ONL were represented by histograms expressed as means±SD (vertical bars). Fluorescence in the ONL showed a significant increase of intensities when using any current application condition as compared to injection without iontophoresis (P<0.05) or no treatment (P<0.05) (*, **). ONL fluorescence showed a significant increase of intensities when using cathodal saline iontophoresis as compared to anodal saline iontophoresis (P<0.05) (**). ONL fluorescence showed a significant increase of intensities when using cathodal saline iontophoresis performed immediately before ODN injection as compared to immediately after ODN injection (P<0.05) (**).

FIG. 6 depicts the analysis of the iontophoresis intensity on the penetration of ODNs in the ONL. Relative fluorescent intensities in ONL were represented by histograms expressed as means±SD (vertical bars). Fluorescence in the ONL shows a significant increase of intensities when using cathodal saline iontophoresis at 1.5 mA as compared to cathodal saline iontophoresis at 0.5 mA (P<0.05) (*).

FIG. 7 depicts the analysis of the iontophoresis effect duration on the ODNs delivery to the ONL. Cathodal saline iontophoresis was performed immediately, 1 hour, 3 hours or 6 hours before the intravitreal injection of labeled ODNs in PN7 rd1/rd1 mice. One hour after the injection, the penetration of ODNs in ONL was quantified. The relative fluorescent intensities in ONL were represented by histograms expressed as means±SD (vertical bars).

FIG. 8 depicts the analysis of the penetration of ODNs to retinal cells at different time point after injection of labeled ODNs with prior saline iontophoresis. Hex-labeled ODNs in red of PN7 rd1/rd1 eye sections at 1 hour (panel A), 4 hours (panel B), 6 hours (panel C), 8 hours (panel D) and 24 hours (panel E) after treatment (cathodal saline iontophoresis immediately prior to intravitreous injection). DAPI staining in blue of top panels is shown in corresponding middle panel (F-J). Double staining with DAPI in blue and Hex-labeled ODNs is shown in corresponding lower panel (K-O). Inset shows a high magnification picture of the ONL with double staining. ONL: outer nuclear layer, INL: inner nuclear layer, GCL: ganglion cell layer. Scale bars: A, B, C, D and E (×25), 100 μm; insets, 10 μm.

FIG. 9 provides photomicrographs of eye sections from PN7 rd1/rd1 mice at different time point after saline iontophoresis. Hematoxylin and eosin stained eye sections from PN7 rd1/rd1 mice showing integrity of the eye structures at 1 hour (Panel A), 6 hours (Panel B) and 24 hours (Panel C) after treatment (cathodal saline iontophoresis immediately prior to intravitreous injection). Higher magnifications showing retina structures from eyes (A), (B) and (C) are shown in (a), (b) and (c) respectively. ONL: outer nuclear layer, INL: inner nuclear layer, GCL: ganglion cell layer. Scale bars: A, B, and C (×2.5), 1 mm; a, b, and c (×25), 100 μm.

FIG. 10 provides photomicrographs of eye semi-thin eye sections at different time points after injection of labeled ODNs with or without prior saline iontophoresis. Semi-thin section of control untreated PN7 rd1/rd1 eye section (Panel A). PN7 rd1/rd1 eye section at 1 hour after intravitreous injection of labeled ODNs without prior saline iontophoresis (Panel B) or with prior cathodal saline iontophoresis (Panel C). Higher magnification showing retina structure from eyes (B) and (C) are shown in (b) and (c) respectively. PN7 rd1/rd1 eye section at 24 hours after intravitreous injection of labeled ODNs without prior saline iontophoresis (D) or with prior cathodal saline iontophoresis (E). Arrow represents vacuoles. RPE: retinal pigment epithelium cells, ONL: outer nuclear layer, INL: inner nuclear layer. Scale bars: A, B, C, D, and E (×25), 50 μm; b, and c, 25 μm.

FIG. 11 provides photomicrographs of ultra-thin eye sections observed by TEM at different time point after injection of labeled ODNs with or without prior saline iontophoresis. Ultra-thin section of control untreated PN7 rd1/rd1 eye section (Panel A). PN7 rd1/rd1 eye section at 1 hour after intravitreous injection of labeled ODNs without prior saline iontophoresis (Panel B) or with prior cathodal saline iontophoresis (Panel C). PN7 rd1/rd1 eye section at 24 hours after intravitreous injection of labeled ODNs without prior saline iontophoresis (Panel D) or with prior cathodal saline iontophoresis (Panel E). Arrow represents vacuoles. RPE: retinal pigment epithelium cells, IS: inner segments, ONL: outer nuclear layer. Scale bars: A, B, C, and D (×25), 5 μm.

FIG. 12 depicts the iontophoresis device and eye sections from PN7 rd1/rd1 mice one hour after treatment. Iontophoresis device: (Panel A) an eye-glass-shaped electrode made with aluminum foil and single-use disposable medical grade hydrophilic polyurethane sponge, (Panel B) iontophoresis generator and the return electrode. Eye section one hour after transpalpebral iontophoresis: (Panel C) hematoxylin and eosin stained eye section showing integrity of the eye structures after iontophoresis (inset: high magnification). Eye sections one hour after intravitreal injection of CY3 tagged ODN: (Panel D) without prior iontophoresis, (Panel E) with prior iontophoresis (inset: high magnification of the ONL with double staining, DAPI stains the nuclei blue and CY3 is red). (Panel F) Control retina from a rd1/rd1 mouse injected with 1 μL of PBS with prior iontophoresis. Scale bars: A, B, 1 cm; C, 1 mm; D, E, F and inset C, 100 μm; inset E, μm.

FIG. 13 provides photomicrographs of eye sections of treated and control PN28 rd1/rd1 mice and ONL cell counting. Hematoxylin-eosin stained sections of rd1/rd1 eyes showing an increased number of nuclei rows in the ONL of ODN-treated eye (arrows): (Panel A) untreated mouse, (Panel B) PBS-treated mouse, (Panel C) WTAS ODN-treated mouse, (Panel D) WTS ODN-treated mouse. (Panel E) Counting of nuclei in the ONL shows a significant increase of nuclei in WTS ODN-treated compared to PBS-treated eyes (P<0.05) and untreated eyes (P<0.01) (*). ONL (Outer Nuclear Layer). Scale bars: A, B, C and D, 100 μm.

FIG. 14 depicts rhodopsin immunohistochemistry on wild-type eye section and rd1/rd1 whole flat-mount retinas, reflecting the time course of the retinal degeneration and the treatment efficacy. (Panel A) Wild-type eye section from a mouse at PN28. (Panel B) Control eye section from wild-type mouse at PN28 using normal mouse serum. (Panel C) rd1/rd1 flat-mount retina from a mouse at PN19 (inset: high magnification). (Panel D) Control flat-mount retina from rd1/rd1 mouse at PN19 using normal mouse serum. (Panel E) rd1/rd1 flat-mount retina from a mouse at PN28. (Panel F) PN28 rd1/rd1 flat-mount retina injected by WTS with prior iontophoresis at PN4, 6, and 8. (Panel G) PN28 rd1/rd1 flat-mount retina injected by WTS without prior iontophoresis at PN4, 6, and 8. (Panel H) PN28 rd1/rd1 flat-mount retina iontophorized without ODN injection at PN4, 6, and 8. (Panel I) PN28 rd1/rd1 flat-mount retina injected with WTSscr7 with prior iontophoresis at PN4, 6, and 8. Scale bars: A and B, 100 μm; C, D, E, F, G, H and I, 1 mm; inset, 10 μm.

FIG. 15 depicts the responsiveness of rhodopsin immunoreactivity to the number of ODN treatment. (Panel A) Three treatments with PBS (PN 4, 6, and 8). (Panel B) One treatment with ODN at PN 4. (Panel C) Two treatments with ODN (PN 4 and 6). (Panel D) Three treatments with ODN (PN 4, 6, and 8). Scale bars: A, B, C and D, 1 mm.

FIG. 16 depicts rhodopsin immunohistochemistry on eye sections from PBS- or ODN-treated rd1/rd1 mice at PN28. (Panel A) DAPI staining in blue and rho-4D2 immunostaining in green (arrows) on section from PN28 PBS-treated rd1/rd1 retina. (Panel B) DAPI staining in blue and rho-4D2 immunostaining in green (arrows) on section from PN28 ODN-treated rd1/rd1 retina. Scale bars: A and B, 150 μm.

FIG. 17 depicts β-PDE immunohistochemistry and western blot. (Panel A) Wild-type +/+ eye section from a mouse at PN 12. (Panel B) Control wild-type +/+ eye section from a mouse at PN 12 using normal rabbit sera. (Panel C) Wild-type +/+ eye section from a mouse at PN 28. (Panel D) Control wild-type +/+ eye section from a mouse al PN 28 using normal rabbit sera. ONL (Outer Nuclear Layer), INL (Inner Nuclear Layer). (Panel E) Anti-β-PDE Western blot: lane 1 is the molecular weight marker (sizes given on left), lane 2 is the polypeptide antigen against which the antibody was grown, lane 3 is protein from a C3H (rd1/rd1) mouse retina lane 4 is protein from a rd1/rd1 mouse retina, lane 5 is protein from an FVB (rd1/rd1) mouse retina, lane 6 is protein from a CCRC (wild-type +/+) mouse retina, lane 7 is protein from a Baln/C (wild-type +/+) mouse retina. Scale bars: A, B, C and D, 100 μm.

FIG. 18 depicts rhodopsin and β-PDE immunohistochemistry on eye sections from PBS- or ODN-treated rd1/rd1 mice at PN28. (Panel A) DAPI staining in blue and β-PDE immunostaining in red (arrows) on section from PN28 ODN-treated rd1/rd1 retina. (Panel B) Combined fluorescence of β-PDE immunostaining in red, rho-4D2 immunostaining in green and DAPI staining in blue on section from PN28 ODN-treated rd1/rd1 retina. Scale bars: A, 150 μm; B, 10 μm.

FIG. 19 depicts representative plots of allele-specific real time PCR. The graph shows the real-time detection of fluorescence resulting from intercalation of SybrGreen fluorescent dye into double-stranded PCR products. Template DNA was isolated from BALB/c mouse (WT), retinas of rd1/rd1 mice treated with WTS ODN (ODN-treated), or retinas of rd1/rd1 mice treated with PBS (PBS-treated). Primers were specific for wild-type allele. Each experimental sample was assayed in 5-10 replicates.

FIG. 20 depicts photoreceptors targeting was significantly increased when the saline transpalpebral iontophoresis is applied before ODN injection as compared with its application after ODN injection.

FIG. 21 shows the results of iontophoresis in target retinal cells

FIG. 22 shows specific coding phosphorothioate oligonucleotide showing a dose-dependant rescue of photoreceptors.

FIG. 23 eye sections from mice after treatment (shows β-phosphodiesterase protein was detected in the eye section).

FIG. 24 is a qualitative evaluation of iontophoresis of labeled oligonucleotide on rat.

FIG. 25 shows reactions with wild-type β-PDE DNA reached threshold in many fewer cycles than those with untreated rd template. A SRT-PCR analysis shows statistically significant leftward shift in the reaction profiles of ODN-treated samples, indicating that treatment induces repair of genomic DNA.

### DETAILED DESCRIPTION OF THE INVENTION

The present invention provides devices and methods for the enhanced delivery of a nucleic acid to tissues of the eye. The methods of the present invention can be used to deliver various different types of nucleic acids to various tissues of the eye. These nucleic acids can be used as diagnostics or therapeutics.

### Nucleic Acids

The term “nucleic acid” is a term of art that refers to a polymer containing at least two nucleotides. “Nucleotides” contain a sugar deoxyribose (in DNA) or ribose (in RNA), a base, and a phosphate group. Nucleotides are linked together through the phosphate groups. “Bases” include purines and pyrimidines, which further include natural compounds adenine, thymine, guanine, cytosine, uracil, inosine, and synthetic derivatives of purines and pyrimidines, or natural analogs.

The term “nucleic acid” also encompasses nucleic acids containing known nucleotide analogs, modified nucleotide (or modified nucleoside or modified base) or modified backbone residues or linkages, which are synthetic, naturally occurring, and non-naturally occurring, which have similar binding properties as the reference nucleic acid.

In the present specification, the term “nucleic acid” is also understood to mean an isolated natural, or a synthetic, a DNA and/or RNA fragment comprising natural and/or non-natural nucleotides, designating a precise succession of at least 10, 15, 20, 25, 30, 35, 40, 45, 50, 75, 100 nucleotides, optionally modified.

The term “modified nucleotide” or “modified nucleoside” or “modified base” refer to variations of the standard bases, sugars and/or phosphate backbone chemical structures occurring in ribonucleic (i.e., A, C, G and U) and deoxyribonucleic (i.e., A, C, G and T) acids. For example, Gm represents 2′-methoxyguanylic acid, Am represents 2′-methoxyadenylic acid, Cf represents 2′-fluorocytidylic acid, Uf represents 2′-fluorouridylic acid, Ar, represents riboadenylic acid. The oligonucleotide can include cytosine or any cytosine-related base including 5-methylcytosine, 4-acetylcytosine, 3-methylcytosine, 5-hydroxymethyl cytosine, 2-thiocytosine, 5-halocytosine (e.g., 5-fluorocytosine, 5-bromocytosine, 5-chlorocytosine, and 5-iodocytosine), 5-propynyl cytosine, 6-azocytosine, 5-trifluoromethylcytosine, N4, N4-ethanocytosine, phenoxazine cytidine, phenothiazine cytidine, carbazole cytidine or pyridoindole cytidine. Modifications can further include guanine or any guanine-related base including 6-methylguanine, 1-methylguanine, 2,2-dimethylguanine, 2-methylguanine, 7-methylguanine, 2-propylguanine, 6-propylguanine, 8-haloguanine (e.g., 8-fluoroguanine, 8-bromoguanine, 8-chloroguanine, and 8-iodoguanine), 8-aminoguanine, 8-sulfhydrylguanine, 8-thioalkylguanine, 8-hydroxylguanine, 7-methylguanine, 8-azaguanine, 7-deazaguanine or 3-deazaguanine. The oligonucleotide can further include adenine or any adenine-related base including 6-methyladenine, N6-isopentenyladenine, N6-methyladenine, 1-methyladenine, 2-methyladenine, 2-methylthio-N6-isopentenyladenine, 8-haloadenine (e.g., 8-fluoroadenine, 8-bromoadenine, 8-chloroadenine, and 8-iodoadenine), 8-aminoadenine, 8-sulfhydryladenine, 8-thioalkyladenine, 8-hydroxyladenine, 7-methyladenine, 2-haloadenine (e.g., 2-fluoroadenine, 2-bromoadenine, 2-chloroadenine, and 2-iodoadenine), 2-aminoadenine, 8-azaadenine, 7-deazaadenine or 3-deazaadenine. Also included is uracil or any uracil-related base including 5-halouracil (e.g., 5-fluorouracil, 5-bromouracil, 5-chlorouracil, 5-iodouracil), 5-(carboxyhydroxylmethyl)uracil, 5-carboxymethylaminomethyl-2-thiouracil, 5-carboxymethylaminomethyluracil, dihydrouracil, 1-methylpseudouracil, 5-methoxyaminomethyl-2-thiouracil, 5′-methoxycarbonylmethyluracil, 5-methoxyuracil, 5-methyl-2-thiouracil, 2-thiouracil, 4-thiouracil, 5-methyluracil, uracil-5-oxyacetic acid methylester, uracil-5-oxyacetic acid, pseudouracil, 5-methyl-2-thiouracil, 2-thiouracil, 3-(3-amino-3-N2-carboxypropyl)uracil, 5-methylaminomethyluracil, 5-propynyl uracil, 6-azouracil, or 4-thiouracil. Examples of other modified base variants known in the art include, without limitation, those listed at 37 C.F.R. §1.822(p)(1), e.g., 4-acetylcytidine, 5-(carboxyhydroxylmethyl)uridine, 2′-methoxycytidine, 5-carboxymethylaminomethyl-2-thioridine, 5-carboxymethylaminomethyluridine, dihydrouridine, 2′-O-methylpseudouridine, β-D-galactosylqueosine, inosine, N6-isopentenyladenosine, 1-methyladenosine, 1-methylpseudouridine, 1-methylguanosine, 1-methylinosine, 2,2-dimethylguanosine, 2-methyladenosine, 2-methylguanosine, 3-methylcytidine, 5-methylcytidine, N6-methyladenosine, 7-methylguanosine, 5-methylaminomethyluridine, 5-methoxyaminomethyl-2-thiouridine, β-D-mannosylqueosine, 5-methoxycarbonyl-methyluridine, 5-methoxyuridine, 2-methylthio-N6-isopentenyladenosine, N-((9-β-D-ribofuranosyl-2-methylthiopurine-6-yl)carbamoyl)threonine, N-((9-β-D-ribofuranosylpurine-6-yl)N-methyl-carbamoyl)threonine, urdine-5-oxyacetic acid methylester, uridine-5-oxyacetic acid (v), wybutoxosine, pseudouridine, queosine, 2-thiocytidine, 5-methyl-2-thiouridine, 2-thiouridine, 4-thiouridine, 5-methyluridine, N-((9-β-D-ribofuranosylpurine-6-yl)carbamoyl)threonine, 2′-O-methyl-5-methyluridine, 2′-O-methyluridine, wybutosine, 3-(3-amino-3-carboxypropyl)uridine. Nucleotides also include any of the modified nucleobases described in U.S. Pat. Nos. 3,687,808, 3,687,808, 4,845,205, 5,130,302, 5,134,066, 5,175,273, 5,367,066, 5,432,272, 5,457,187, 5,459,255, 5,484,908, 5,502,177, 5,525,711, 5,552,540, 5,587,469, 5,594,121, 5,596,091, 5,614,617, 5,645,985, 5,830,653, 5,763,588, 6,005,096 and 5,681,941. Examples of modified nucleoside and nucleotide sugar backbone variants known in the art include, without limitation, those having, e.g., 2′ ribosyl substituents such as F, SH, SCH3, OCN, Cl, Br, CN, CF3, OCF3, SOCH3, SO2, CH3, ONO2, NO2, N3, NH2, OCH2CH₂OCH3, O(CH2)2ON(CH3)2, OCH2OCH2N(CH3)2, O(C1-10 alkyl), O(C2-10 alkenyl), O(C2-10 alkynyl), S(C1-10 alkyl), S(C2-10 alkenyl), S(C2-10 alkynyl), NH(C1-10 alkyl), NH(C2-10 alkenyl), NH(C2-10 alkynyl), and O-alkyl-O-alkyl. Desirable 2′ ribosyl substituents include 2′-methoxy (2′-OCH3), 2′-aminopropoxy (2′ OCH2CH2CH2NH2), 2′-allyl (2′-CH2-CH═CH2), 2′-O-allyl (2′-O—CH2-CH═CH2), 2′-amino (2′—NH2), and 2′-fluoro (2′-F). The 2′-substituent may be in the arabino (up) position or ribo (down) position.

The term “nucleic acid” also encompasses nucleic acids containing 5′-5′ and 3′-3′ inverted nucleotide caps. As used herein, the term “5′-5′ inverted nucleotide cap” means a first nucleotide covalently linked to the 5′ end of an oligonucleotide via a phosphodiester linkage between the 5′ position of the first nucleotide and the 5′ terminus of the oligonucleotide as shown below.

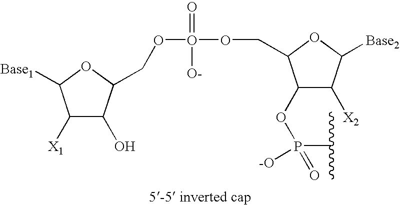

The term “3′-3′ inverted nucleotide cap” is used herein to mean a last nucleotide covalently linked to the 3′ end of an oligonucleotide via a phosphodiester linkage between the 3′ position of the last nucleotide and the 3′ terminus of the oligonucleotide as shown below.

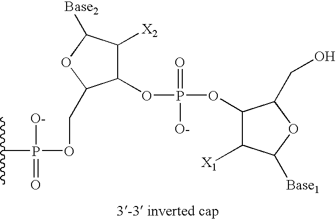

Further modifications include conjugation at either the 5′ or 3′ end with; polyethylene glycol, polyesters, polyanyhdrides, polycarbonates, polyurethanes, methacrylates, lipids, biopolymers (hyaluronic acid, cellulose derivatives, chitosan, alginate, etc.), thermoreponsive polymers (pluronix), dendrimers, poly-amines, proteins, poly-peptides, antibodies, metals (gold, silver, etc.), chelating agents, molecules to aid in detection (fluorophores and chromophores).

In the present specification, the term “nucleic acid” is also understood to include nucleic acid molecules that modulate the expression or function of one or more target genes including, but not limited to, antisense and enzymatic nucleic acid molecules, such as hammerhead ribozymes, DNAzymes, allozymes, aptamers, decoys and siRNA (RNAi).

Oligonucleotide agents have been shown to have functional activity in vitro and thus the promise of therapeutic potential. High sensitivity to nuclease digestion, however, makes oligonucleotide agents unstable and thus impracticable for in vivo administration. However, methods for stabilizing nucleic acid or oligonucleotides have been developed in the art that can be used to produce high binding, nuclease-resistant oligonucleotide that retain their specificity. In one example, U.S. Pat. No. 6,423,493, issued Jul. 23, 2002 discloses a random combinatorial selection method is disclosed for the construction of oligonucleotide aptamers in which nuclease resistance is conferred by the inclusion of modified nucleotides. The modified nucleotides are incorporated during PCR amplification to form achiral modified oligonucleotides. In another example, mRNAs are stabilized by modifying the sequence and optimizing for translation. See US Patent Application Nos. 2005/0032730 A1 and 2005/0250723 A1. In the present specification, the term “nucleic acid” is also understood to include stabilized nucleic acid molecules.

The oligonucleotide(s) of the present invention can be incorporated into pharmaceutical compositions suitable for administration. The pharmaceutical compositions generally comprise at least one oligonucleotide and a pharmaceutically-acceptable carrier in a form suitable for administration to a subject. Pharmaceutically-acceptable carriers are determined in part by the particular composition being administered, as well as by the particular method used to administer the composition. Accordingly, there is a wide variety of suitable formulations of pharmaceutical compositions for administering the oligonucleotide compositions (Remington's Pharmaceutical Sciences, Mack Publishing Co., Easton, Pa. 18th ed., 1990). The pharmaceutical compositions are generally formulated as sterile, substantially isotonic and in full compliance with all Good Manufacturing Practice (GMP) regulations of the U.S. Food and Drug Administration.

The terms “pharmaceutically-acceptable,” “physiologically-tolerable,” and grammatical variations thereof, as they refer to compositions, carriers, diluents and reagents, are used interchangeably and represent that the materials are capable of administration to or upon a subject without the production of undesirable physiological effects to a degree that would prohibit administration of the composition. For example, “pharmaceutically-acceptable excipient” means an excipient that is useful in preparing a pharmaceutical composition that is generally safe, non-toxic, and desirable, and includes excipients that are acceptable for veterinary use as well as for human pharmaceutical use. Such excipients can be solid, liquid, semisolid, or, in the case of an aerosol composition, gaseous. “Pharmaceutically-acceptable salts and esters” means salts and esters that are pharmaceutically-acceptable and have the desired pharmacological properties. Such salts include salts that can be formed where acidic protons present in the oligonucleotide are capable of reacting with inorganic or organic bases. Suitable inorganic salts include those formed with the alkali metals, e.g., sodium and potassium, magnesium, calcium, and aluminum. Suitable organic salts include those formed with organic bases such as the amine bases, e.g., ethanolamine, diethanolamine, triethanolamine, tromethamine, N methylglucamine and the like. Such salts also include acid addition salts formed with inorganic acids (e.g., hydrochloric and hydrobromic acids) and organic acids (e.g., acetic acid, citric acid, maleic acid, and the alkane- and arene-sulfonic acids such as methanesulfonic acid and benzenesulfonic acid). Pharmaceutically-acceptable esters include esters formed from carboxy, sulfonyloxy, and phosphonoxy groups present in the oligonucleotide, e.g., C1-6 alkyl esters. Where there are two acidic groups present, a pharmaceutically-acceptable salt or ester can be a mono-acid-mono-salt or ester or a di-salt or ester; and similarly where there are more than two acidic groups present, some or all of such groups can be salified or esterified. The oligonucleotide of the invention can be present in unsalified or unesterified form, or in salified and/or esterified form, and the naming of such oligonucleotide is intended to include both the original (unsalified and unesterified) compound and its pharmaceutically-acceptable salts and esters. A person of ordinary skill in the art would have no difficulty determining the appropriate timing, sequence and dosages of administration for particular drugs and compositions of the present invention.

Examples of such carriers or diluents include, but are not limited to, water, saline, Ringer's solutions, dextrose solution, and 5% human serum albumin. Liposomes and non-aqueous vehicles such as fixed oils can also be used. The use of such media and compounds for pharmaceutically active substances is well known in the art. Except insofar as any conventional media or compound is incompatible with the oligonucleotide, use thereof in the compositions is contemplated. Supplementary active compounds can also be incorporated into the compositions.

A pharmaceutical composition of the invention is formulated to be compatible with its intended route of administration. In some methods, the oligonucleotide of the invention are administered as a sustained release composition or device, such as a Medipad™ device.

The oligonucleotide of the invention can optionally be administered in combination with other agents that are at least partly effective in treating various diseases including various oligonucleotide-related diseases.

Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial compounds such as benzyl alcohol or methyl parabens; antioxidants such as ascorbic acid or sodium bisulfite; chelating compounds such as ethylenediaminetetraacetic acid (EDTA); buffers such as acetates, citrates or phosphates, and compounds for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

In all cases, the composition must be sterile. It should be stable under the conditions of manufacture and storage and should be preserved against the contaminating action of microorganisms such as bacteria and fungi. The carrier can be a solvent or dispersion medium containing, e.g., water, ethanol, polyol (e.g., glycerol, propylene glycol, and liquid polyethylene glycol, and the like), and suitable mixtures thereof. The proper fluidity can be maintained, e.g., by the use of a coating such as lecithin, by the maintenance of the required particle size in the case of dispersion and by the use of surfactants. Prevention of the action of microorganisms can be achieved by various antibacterial and antifungal compounds, e.g., parabens, chlorobutanol, phenol, ascorbic acid, thimerosal, and the like. In many cases, it will be preferable to include isotonic compounds, e.g., sugars, polyalcohols such as manitol, sorbitol, sodium chloride in the composition. Prolonged absorption of the injectable compositions can be brought about by including in the composition a compound that delays absorption, e.g., aluminum monostearate and gelatin.

Sterile solutions can be prepared by incorporating the oligonucleotide in the required amount in an appropriate solvent with one or a combination of ingredients enumerated above, as required, followed by filtered sterilization. Generally, dispersions are prepared by incorporating the binding agent into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Administration can also be by transmucosal or transdermal means. For occular administration by iontophoretic administration, penetrants appropriate to the barrier to be permeated are used in the formulation. Such penetrants are generally known in the art, and include, e.g., for transmucosal administration, detergents, bile salts, and fusidic acid derivatives. The oligonucleotide is formulated into ointments, salves, gels, or creams as generally known in the art.

In one embodiment, the oligonucleotide is prepared with carriers that will protect the oligonucleotide against rapid elimination, such as a controlled-release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art.

The nucleic acid molecules of the invention can be inserted into vectors and used as gene therapy vectors. Gene therapy vectors can be delivered to a subject by, e.g., intravenous injection, local administration (U.S. Pat. No. 5,328,470) or by stereotactic injection (Chen et al., Proc. Natl. Acad. Sci. USA, 91:3054-3057, 1994). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells that produce the gene delivery system. The pharmaceutical compositions can be included in a container, pack, or dispenser together with instructions for administration.

A particular nucleic acid sequence also implicitly encompasses “splice variants.” Similarly, a particular protein encoded by a nucleic acid implicitly encompasses any protein encoded by a splice variant of that nucleic acid. “Splice variants,” as the name suggests, are products of alternative splicing of a gene. After transcription, an initial nucleic acid transcript can be spliced such that different (alternate) nucleic acid splice products encode different polypeptides. Mechanisms for the production of splice variants vary, but include alternate splicing of exons. Alternate polypeptides derived from the same nucleic acid by read-through transcription are also encompassed by this definition. Any products of a splicing reaction, including recombinant forms of the splice products, are included in this definition.

A liquid aqueous medium or other material is ophthalmically acceptable when it is compatible with ocular tissue, that is, it does not cause significant or undue detrimental effects when brought into contact with ocular tissue. An ophthalmic composition or pharmaceutical composition of the present invention is a composition that is compatible with ocular tissue, e.g., a composition that is suitable for administration to the eye.

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all.

“Pharmaceutically acceptable excipient” means an excipient that is useful in preparing a pharmaceutical composition that is generally safe, non-toxic, and desirable, and includes excipients that are acceptable for veterinary use as well as for human pharmaceutical use. Such excipients can be solid, liquid, semisolid, or, in the case of an aerosol composition, gaseous.

A “therapeutically effective amount” means the amount that, when administered to a subject for treating a disease, is sufficient to effect treatment for that disease.

Except when noted, the terms “subject” or “patient” are used interchangeably and refer to mammals such as human patients and non-human primates, as well as experimental animals such as rabbits, dogs, cats, rats, mice, and other animals. Accordingly, the term “subject” or “patient” as used herein means any mammalian patient or subject to which the compositions of the invention can be administered. In some embodiments of the present invention, the patient will be suffering from a condition that causes lowered resistance to disease, e.g., HIV. In an exemplary embodiment of the present invention, to identify subject patients for treatment with a pharmaceutical composition comprising one or more collectins and/or surfactant proteins according to the methods of the invention, accepted screening methods are employed to determine the status of an existing disease or condition in a subject or risk factors associated with a targeted or suspected disease or condition. These screening methods include, for example, ocular examinations to determine whether a subject is suffering from an ocular disease. These and other routine methods allow the clinician to select subjects in need of therapy. In certain embodiments of the present invention, ophthalmic compositions for storing, cleaning, re-wetting and/or disinfecting a contact lens, as well as artificial tear compositions and/or contact lenses will contain one or more collectins and/or surfactant proteins thereby inhibiting the development of ocular disease in contact-lens wearers.

Iontophoresis of Oligonucleotides

The movement of molecules in a current induced field is dependent on several factors among which size, hydrophilicity and ionic state are paramount. Within a homologous series of compounds where these factors are kept relatively constant, one can reasonably expect the iontophoretic mobility of each member of the series to be similar. Oligonucleotides represent such a series of compounds.

There are a variety of devices that have been disclosed (part of them are available on the market) (see for example the patent documents: U.S. Pat. No. 4,141,359, issued Feb. 27, 1979; U.S. Pat. No. 4,250,878, issued Jan. 17, 1981; U.S. Pat. No. 4,301,794, issued Nov. 24, 1981; U.S. Pat. No. 4,747,819, issued Apr. 31, 1988; U.S. Pat. No. 4,752,285, issued Jun. 21, 1988; U.S. Pat. No. 4,915,685, issued Apr. 10, 1990; U.S. Pat. No. 4,979,938, issued Dec. 25, 1990; U.S. Pat. No. 5,252,022, issued Oct. 5, 1993; U.S. Pat. No. 5,374,245, issued Dec. 20, 1994; U.S. Pat. No. 5,498,235, issued Mar. 12, 1996; U.S. Pat. No. 5,730,716, issued Mar. 24, 1998; U.S. Pat. No. 6,001,088, issued Dec. 14, 1999; U.S. Pat. No. 6,018,679, issued Jan. 25, 2000; U.S. Pat. No. 6,139,537, issued Oct. 31, 2000; U.S. Pat. No. 6,148,231, issued Nov. 14, 2000; U.S. Pat. No. 6,154,671, issued Nov. 28, 2000, and U.S. Pat. No. 6,167,302, issued Dec. 26, 2000).

The typical molecular weight of a therapeutically relevant oligonucleotide is 110 kD. Since each nucleotide is coupled via a phosphor- or thiodiester, each linkage possesses a negative charge. The average molecular weight of a nucleotide is 335 (330 for RNA and 340 for DNA), each oligonucleotide possesses 29 or more negative charges. Any modifications to the sugar moiety (2′ modifications) does not diminish the charge density and any increase in lipophilicity brought about by the modification is more than compensated for by this inherent negative charge.

A study by van der Geest et al. (Pharm. Res., 13:553-558, 1996) examined the in vitro iontophoretic mobility of two representative bases: uracil (RNA) and adenine (DNA); two representative nucleosides: Uridine (RNA) and Adenosine (DNA); and representative nucleotides: AMP, ATP, GTP and imido-GTP across mammalian skin. They report that the efficiency of delivery was weakly dependent upon any differences in water solubility, inversely sensitive to molecular weight, and strongly influenced by charge.

An additional study by Brand et al. (J. Pharm. Sci., 87:49-52, 1998) further investigates the effects of size and sequence on iontophoretically assisted transdermal delivery. The studies included oligonucleotides ranging from 6-40 bases (2 kD-13.5 kD) and composed of various nucleotide units with differing repeat units. They observed differences in the transdermal flux levels with certain repeating base units. It is known that tetrameric sequences of guanidine form stable G-quartets. These quartets have large surface areas and the authors suggest that the increase in molecular radius impacts the flux rate. This increase may very well be important to transdermal delivery where the stratum corneum limits passive delivery of compounds with a molecular weight under 500.

The sclera has been shown to passively allow transscleral delivery of proteins and polysaccharides with molecular weights up to 150 kD. Also, Asahara demonstrated the transcorneal delivery of a 4.7 kB plasmid (˜174 kD). The eye has clearly been demonstrated to be exceedingly more permeable to therapeutics than the skin. Any observed retardation of flux derived from sequence specific secondary structures within an oligonucleotide series in the skin would not be a factor in ocular tissue. One could reasonably expect that the main factor governing iontophoretic mobility would be charge density, regardless of the specific sequence.

The enhanced retinal delivery of oligonucleotides is demonstrated in Example 1 utilizing a chimeric oligonucleotide. A chimeric oligonucleotide, being composed of both RNA and DNA nucleotides, represents an all encompassing example of the types of oligonucleotides one would deliver. The successful delivery of the chimeric oligonucleotide would lead one skilled in the art to predict the successful delivery of a wide range of oligonucleotides belonging to the subcategories of: aptamers, antisense, siRNA, etc. (Jayasena, S., Clinical Chemistry, 45:1628-1650, 1999; Henry, S. et al., Exp. Opin. Pharmacother., 2:1-15, 2001; Marro, D. et al., Drug Delivery Pharm. Res., 18:1701-1708, 2001).

The present invention is also directed to methods wherein a nucleic acid comprised in a composition of the invention is capable of specifically hybridizing with part of target nucleic acid, preferably a target gene (genomic DNA), or target protein belonging to said target cells. Among the nucleic acids that can delivered by the method of the present invention are oligonucleotide sense or antisense or a triple helix capable of modulating the expression products of a target gene of cells can be cited, in addition to the oligo- or polynucleotide (DNA or RNA) or the chimeric oligonucleotides relating to the correction of a functionally deficient gene, or to the creation of a deficient gene disclosed in the above cited documents or in the present specification, as below.

“Specifically hybridizing” is a term that is used to indicate a sufficient degree of complementarity such that stable and specific binding occurs between the nucleic acid target, DNA or RNA target, and the nucleic acid that can delivered by the method of the present invention.

In a further preferred embodiment, the invention relates to a method wherein a nucleic acid, particularly an oligo- or polynucleotide (DNA or RNA) or a chimeric oligonucleotide as defined above, comprised in a composition is a polynucleotide containing at least a sequence complementary to a target gene of cells with the exception of at least one nucleotide that is desired to be inserted, or deleted or substituted in said target gene.

“A sequence complementary to a target gene” means a sequence forming Watson-Crick base pairing with part of the target gene sequence, part of the target gene sequence that particularly comprises, in the context of this invention, the fragment of the target sequence wherein said at least one nucleotide is desired to be inserted (or deleted) or changed. Guanine/cytosine or adenine/thymine (or /uracil) are examples of complementary bases that are known to form hydrogen bonds between them.

The term “chimeric oligonucleotide” is defined as a polynucleotide having both ribonucleotides, modified or not and deoxyribonucleotides in a first strand and solely deoxyribonucleotides in a second strand wherein the strands have a Watson-Crick complementarity and are linked by oligonucleotides so that the polynucleotide has at most a single 3′ and a single 5′ end, and wherein these ends can be ligated so that the polynucleotide is a single continuous circular polymer.

Nucleotides are the monomeric units of nucleic acid polymers. A “polynucleotide” is distinguished here from an “oligonucleotide” by containing more than 80 monomeric units; oligonucleotides contain from 2 to 80 nucleotides. The term nucleic acid includes deoxyribonucleic acid (“DNA”) and ribonucleic acid (“RNA”). DNA can be in the form of antisense, plasmid DNA, parts of a plasmid DNA, expression vectors, expression cassettes, chimeric sequences, chromosomal DNA, or derivatives of these groups. RNA can be in the form of oligonucleotide RNA, tRNA (transfer RNA), snRNA (small nuclear RNA), rRNA (ribosomal RNA), mRNA (messenger RNA), a shRNA (short RNA), miRNA (microRNA), a siRNA (small interfering RNA), antisense RNA, ribozymes, chimeric sequences, or derivatives of these groups.

In addition, DNA and RNA can be single, double, triple, or quadruple stranded, in a linear or circular form and eventually closed.

“Antisense” is a nucleic acid that interferes with the function of DNA and/or RNA. This may result in suppression of expression. Natural nucleic acids have a phosphate backbone, artificial nucleic acids may contain other types of backbones, nucleotides, or bases. These include PNAs (peptide nucleic acids), phosphothioates, and other variants of the phosphate backbone of native nucleic acids such as phosphoramidates, methyl phosphonates, chiral-methyl phosphonates, 2-O-methyl ribonucleotides, peptide-nucleic acids (PNAs). So, by antisense nucleic acid molecules, it is intended to designate molecules that are complementary to the sense targeting nucleic acid or to the sense nucleic encoding the target protein (such as complementary to the coding strand of a double stranded cDNA molecule or complementary to an mRNA sequence). Accordingly, an antisense nucleic acid can hydrogen bond to a sense nucleic acid. The antisense nucleic acid can be complementary to an entire protein coding strand, or to only a portion thereof, e.g., all or part of the protein coding region (or open reading frame). An antisense nucleic acid molecule can be antisense to a non-coding region of the coding strand of a nucleotide sequence encoding the target protein. An antisense oligonucleotide can be, for example, about 5, 10, 15, 20, 25, 30, 35, 40, 45, 50, 60 or 75 nucleotides in length.

The administered nucleic acid of the method of the invention, such as oligonucleotide DNA, oligonucleotide RNA, or chimeric oligonucleotide, can be constructed using chemical synthesis and enzymatic ligation reactions using procedures known in the art. For example, an oligonucleotide or polynucleotide nucleic acid can be chemically synthesized using naturally occurring nucleotides or variously modified nucleotides designed to increase the biological stability of the nucleic acid molecules or to increase the physical stability of the duplex formed between the antisense and sense nucleic acids, e.g., phosphorothioate derivatives. Alternatively, the nucleic acid can be produced biologically using an expression vector into which a nucleic acid has been subcloned.

“Expression cassette” refers to a natural or recombinantly produced nucleic acid that is capable of expressing protein(s). A DNA expression cassette typically includes a promoter (allowing transcription initiation), and a sequence encoding one or more proteins. Optionally, the expression cassette can include transcriptional enhancers, non-coding sequences, splicing signals, transcription termination signals, and polyadenylation signals. An RNA expression cassette typically includes a translation initiation codon (allowing translation initiation), and a sequence encoding one or more proteins. Optionally, the expression cassette may include translation termination signals, a polyadenosine sequence, internal ribosome entry sites (IRES), and non-coding sequences.

The terms siRNA, shRNA, miRNA, like antisense nucleic acid (DNA or RNA), refer to a nucleic acid that has the ability to reduce or inhibit expression (transcription and/or translation) of a target nucleic acid sequences when said nucleic acid is introduced or expressed in the same cell as the target nucleic acid. Said nucleic acid has substantial or complete identity to the complementary sequence of the target gene and can so hybridizes with it. Typically, said nucleic acid is at least about 15-80 nucleotides in length, preferably about 15-50 nucleotides in length, 20-30 nucleotides length is more preferred.

The term “nucleic acid” is also intended to designate ribozymes, which are capable of selectively destroying target RNAs (see the document EP 321 201).

In a further preferred embodiment, the invention relates to a method according to the present invention, wherein said nucleic acid comprised in the composition is an aptamer.

As used herein, the term “aptamer” means any polynucleotide, or salt thereof, having selective binding affinity for a non-polynucleotide molecule (such as a protein) via non-covalent physical interactions. An aptamer is a polynucleotide that binds to a ligand in a manner analogous to the binding of an antibody to its epitope.

Aptamers are chemically synthesized short strands of nucleic acid that adopt specific three-dimensional conformations and are selected for their affinity to a particular target through a process of in vitro selection referred to as systematic evolution of ligands by exponential enrichment (SELEX). SELEX is a combinatorial chemistry methodology in which vast numbers of oligonucleotides are screened rapidly for specific sequences that have appropriate binding affinities and specificities toward any target. Using this process, novel aptamer nucleic acid ligands that are specific for a particular target may be created. The SELEX process in general, and VEGF aptamers and formulations in particular, are described in, e.g., U.S. Pat. Nos. 5,270,163, 5,475,096, 5,696,249, 5,670,637, 5,811,533, 5,817,785, 5,958,691, 6,011,020, 6,051,698, 6,147,204, 6,168,778 and 6,426,335, the content of each of which is specifically incorporated by reference herein. Anti-VEGF aptamers are small stable RNA-like molecules that bind with high affinity to the 165 kDa isoform of human VEGF.

Many other aptamer sequences have been developed that target various other biological targets. For example aptamer sequences have been developed that target PDGF (U.S. Pat. Nos. 5,668,264, 5,674,685, 5,723,594, 6,229,002, 6,582,918 and 6,699,843), basic FGF (U.S. Pat. Nos. 5,459,015 and 5,639,868), CD40 (U.S. Pat. No. 6,171,795), TGFβ (U.S. Pat. Nos. 6,124,449; 6,346,611; and 6,713,616), CD4 (U.S. Pat. No. 5,869,641), chorionic gonadotropin hormone (U.S. Pat. Nos. 5,837,456 and 5,849,890), HKGF (U.S. Pat. Nos. 5,731,424, 5,731,144, 5,837,834 and 5,846,713), ICP4 (U.S. Pat. No. 5,795,721), HIV-reverse transcriptase (U.S. Pat. No. 5,786,462), HIV-integrase (U.S. Pat. Nos. 5,587,468 and 5,756,287), HIV-gag (U.S. Pat. No. 5,726,017), HIV-tat (U.S. Pat. No. 5,637,461), HIV-RT and HIV-rev (U.S. Pat. Nos. 5,496,938 and 5,503,978), HIV nucleocapsid (U.S. Pat. Nos. 5,635,615 and 5,654,151), neutophil elastase (U.S. Pat. Nos. 5,472,841 and 5,734,034), IgE (U.S. Pat. Nos. 5,629,155 and 5,686,592), tachykinin substance P (U.S. Pat. Nos. 5,637,682 and 5,648,214), secretory phospholipase A2 (U.S. Pat. No. 5,622,828), thrombin (U.S. Pat. No. 5,476,766), intestinal phosphatase (U.S. Pat. Nos. 6,280,943, 6,387,635 and 6,673,553), tenascin-C (U.S. Pat. Nos. 6,232,071 and 6,596,491), as well as to cytokines (U.S. Pat. No. 6,028,186), seven transmembrane G protein-coupled receptors (U.S. Pat. No. 6,682,886), DNA polymerases (U.S. Pat. Nos. 5,693,502, 5,763,173, 5,874,557 and 6,020,130,) complement system proteins (U.S. Pat. Nos. 6,395,888 and 6,566,343), lectins (U.S. Pat. Nos. 5,780,228, 6,001,988, 6,280,932 and 6,544,959), integrins (U.S. Pat. No. 6,331,394), and hepatocyte growth factor/scatter factor (HGF/SP) or its receptor (c-met) (U.S. Pat. No. 6,344,321). Still many more aptamers that target a desired biological target are possible given the adaptability of the SELEX-based methodology.

In further embodiments of the invention, the aptamer is directed to an adhesion molecule, such as ICAM-1, or its binding LFA-1. In further embodiments, the aptamer is directed to any known ligand or its receptor. Examples of ligands and/or their receptors for targeting with the sterically-enhanced aptamer conjugates of the invention include TGF, PDGF, IGF, and FGF. Further ligands and/or their receptors for targeting include: cytokines, lymphokines, growth factors, or other hematopoietic factors such as M-CSF, GM-CSF, TNF, IL-1, IL-2, IL-3, IL-4, IL-5, IL-6, IL-7, IL-8, IL-9, IL-10, IL-11, IL-12, IL-13, IL-14, IL-15, IL-16, IL-17, IL18, IFN, TNF0, TNF1, TNF2, G-CSF, Meg-CSF, GM-CSF; thrombopoietin, stem cell factor, and erythropoietin, hepatocyte growth factor/NK1 or factors that modulate angiogenesis, such as angiopoietins Ang-1, Ang-2, Ang-4, Ang-Y, and/or the human angiopoietin-like polypeptide, and/or vascular endothelial growth factor (VEGF). Particular other factors for targeting with the compositions of the invention include angiogenin, BMPs such as bone morphogenic protein-1, etc., bone morphogenic protein receptors such as bone morphogenic protein receptors IA and IB, neurotrophic factors, chemotactic factor, CD proteins such as CD3, CD4, CD8, CD19 and CD20; erythropoietin; osteoinductive factors; immunotoxins; bone morphogenetic proteins (BMPs); interferons, such as interferon-alpha, -beta, and -gamma; colony stimulating factors (CSFs), e.g., M-CSF, GM-CSF, and G-CSF; interleukins (ILs), e.g., IL-1 to IL-10; superoxide dismutase; T-cell receptors; surface membrane proteins; decay accelerating factor; viral antigen such as, for example, a portion of the AIDS envelope; transport proteins; homing receptors; addressins; regulatory proteins; integrins such as CD11a, CD11b, CD11c, CD18, an ICAM, VLA-4 and VCAM; a tumor associated antigen such as HER2, HER3 or HER4 receptor; and fragments and/or variants of any of the above-listed polypeptides.

The invention further includes compositions comprising any of the known aptamer nucleic acid sequences that target, for example, a ligand or its receptor, such as those compiled in the aptamer database, which is available at the website, aptamer.icmb.utexas.edu).

In some embodiments, the nucleic acid molecules used in the present invention can be modified at the base moiety, sugar moiety or phosphate backbone to improve, e.g., the stability, hybridization, or solubility of the molecule. For example, the deoxyribose phosphate backbone of the nucleic acids can be modified to generate peptide nucleic (“PNAs”).

In one embodiment, the invention relates to a method according to the present invention wherein said nucleic acid comprised in the composition is a chimeric oligonucleotide DNA/2′OMeRNA type wherein at least part of that DNA/RNA sequence is complementary to a genomic DNA fragment sequence of a target mutated gene of said cells with the exception of the mutation that is desired to be reverted in said target mutated gene.

In another embodiment, the nucleic acid is an oligonucleotide DNA or an oligonucleotide RNA, optionally with phosphorothioates linkages.

In another embodiment, the nucleic acid is a single stranded oligonucleotide DNA (ssODN) or a single stranded oligonucleotide RNA (ssORN).

### Target Cells

In one embodiment, the nucleic acid is capable of specifically hybridizing with a sequence of a genomic DNA contained in said retinal cells.

In another embodiment, the nucleic acid is capable of specifically hybridizing with a sequence located into the nucleus of said retinal cells.

In another preferred embodiment, the nucleic acid is capable of binding to an extracellular protein, thereby inhibiting the protein binding with its designated receptor.

In another embodiment, the target cells are photoreceptor cells or retinal pigment epithelium cells (RPE cells), photoreceptor cells are preferred.

In another preferred embodiment, the nucleic acid target cells/proteins are located in the outer nuclear layer of the retina.

In a second aspect of the method for delivering in vivo a nucleic acid into target cells of the present invention, the step of injection of said composition comprising said nucleic is selected from the group consisting of periocular (subconjunctival, peribulbar, laterobulbar, subtenon), subretinal, supra choroidal, intracameral, intracorneal, and intravitreous injection of the composition comprising said nucleic acid.

In the method for delivering in vivo a nucleic acid into target cells of the present invention, it is also preferred that the administration of the composition containing the nucleic acid is carried out by a step of topical instillation of said composition. A preferred application would be a topical application at the time of iontophoresis utilizing the device disclosed in the U.S. Pat. No. 6,154,671, issued Nov. 28, 2000.

It has been established that iontophoretically assisted trans-corneal delivery of oligonucleotides was feasible. Ashara, T., et. al. (Jpn. J. Opthamol., 45:31-39, 2001) reported the successful trans-corneal delivery of a 4.7 kB plasmid (˜174 kD) to the anterior chamber and a 23 base pair phosphothioate (˜8 kD) to the posterior chamber. The permeability of the sclera was also well established by a series of experiments by Ambati, J., et al. (IOVS, 41:1186-1191, 2000; IOVS, 41:1181-1191, 2000). It was established that the passive transscleral delivery to the posterior chamber was feasible with IgG and Dextran polymers up to 150 kDa.

In another aspect of the method for delivering in vivo a nucleic acid into target cells of the present invention, the iontophoresis is an ocular or a transpalpebral iontophoresis.

Devices for delivery of therapeutic or diagnostic agents into target cells of an eye tissue through ocular or transpalpebral iontophoresis are commonly used and thus have been already disclosed. The skilled artisan could easily choose and determined the iontophoresis device and its use conditions, particularly the current density, the period of time of applying the current and the electrodes form and location etc., adapted to the tissue containing the target cells where the nucleic acid transfer is desired to be done.

### Ocular Iontophoresis System

In one embodiment, the ocular iontophoresis system used in the methods of the present invention is a device selected in the group consisting of the devices disclosed in the following patents: U.S. Pat. No. 4,141,359, issued Feb. 27, 1979; U.S. Pat. No. 4,250,878, issued Jan. 17, 1981; U.S. Pat. No. 4,301,794, issued Nov. 24, 1981; U.S. Pat. No. 4,747,819, issued Apr. 31, 1988; U.S. Pat. No. 4,752,285, issued Jun. 21, 1988; U.S. Pat. No. 4,915,685, issued Apr. 10, 1990; U.S. Pat. No. 4,979,938, issued Dec. 25, 1990; U.S. Pat. No. 5,252,022, issued Oct. 5, 1993; U.S. Pat. No. 5,374,245, issued Dec. 20, 1994; U.S. Pat. No. 5,498,235, issued Mar. 12, 1996; U.S. Pat. No. 5,730,716, issued Mar. 24, 1998; U.S. Pat. No. 6,001,088, issued Dec. 14, 1999; U.S. Pat. No. 6,018,679, issued Jan. 25, 2000; U.S. Pat. No. 6,139,537, issued Oct. 31, 2000; U.S. Pat. No. 6,148,231, issued Nov. 14, 2000; U.S. Pat. No. 6,154,671, issued Nov. 28, 2000, and U.S. Pat. No. 6,167,302, issued Dec. 26, 2000.

In a preferred embodiment, the ocular iontophoresis system used in the methods of the present invention is a device selected in the group consisting of the devices disclosed in the U.S. Pat. No. 6,154,671, issued Nov. 28, 2000, said device being characterized in that it comprises a reservoir configured to receive an aqueous solution, optionally buffered and including various electrolytes, and having an internal wall, an external wall, and an end wall bridging the internal wall and the external wall, the internal wall and the external wall being annular and having a free end configured to be applied to an eyeball, said device further comprising at least one active electrode arranged in the reservoir, a passive electrode and a current generator, wherein the at least one active electrode is a surface electrode arranged on an interior surface of the end wall and wherein the internal wall has an outer diameter that is configured to be at least equal to a predetermined diameter, whereby the predetermined diameter represents a diameter of a human cornea.

In another preferred embodiment, a transpalpebral iontophoresis system is used in the methods of the present invention is a transpalpebral iontophoresis device disclosed in the U.S. Patent Application No. US 2004/267188 on Dec. 30, 2004. The device comprises a main electrode having an insulating layer and an adhesive layer able to bond the insulating layer to a conductive layer characterized in that the main electrode has an area able to come into contact with an eyelid. The transpalpebral iontophoresis can be an anionic or cationic iontophoresis, although cationic iontophoresis is also preferred for transpalpebral iontophoresis. Transpalpebral iontophoresis is preferably performed with a current of about 1-5 mA for about 1-7 min; more preferably with a current of about 1-3 mA for about 3-6 min; and more preferably with a current of about 2 mA for up to 5 min being more preferred.

### Eye Diseases

In one aspect, the present invention is directed to a method for the treatment of an eye disease resulting from a deficient protein with a therapeutic nucleic acid that is capable of specifically hybridizing with a target DNA or RNA sequence encoding said deficient protein.

In one embodiment, the invention relates to a method of the invention for the treatment of an eye inherited pathology resulting from a mutated gene expressed in the target cells with a nucleic acid composition containing at least a sequence complementary to a genomic DNA sequence of the gene, preferably wherein at least part of the nucleic acid composition is complementary to a genomic DNA sequence of the mutated gene expressed in the target cells, with the exception of the mutation that is desired to be reverted in said target gene.

The present invention also encompasses a method to treat an eye disease with a nucleic acid capable of reverting or inducing a mutation in a gene of target eye cells, gene expression of which is associated to that disease, in a non-human animal or in a human subject in need of such treatment.

In the present specification, the term “mutated gene” is understood to mean a gene whose sequence comprises at least one mutation or polymorphism relative to a wild-type reference. The mutation can be, for example, a deletion, addition or substitution of at least one nucleotide compared to the wild-type gene. A mutation can be at least partially responsible of a pathology or affection, notably associated with the loss of the normal function of the protein encoded by the wild-type functional gene.

Among the target genes that can be treated with the method of the present invention, gene responsible for inherited retinopathies that are a genetically and phenotypically-heterogeneous group of diseases affecting approximately one in 2000 individuals worldwide can be particularly cited (Sohocki et al., Hum. Mutat., 17:42-51, 2001).

Among these target genes, the murine gene encoding the cGMP-phosphodiesterase β-subunit wherein the non-sense C to A mutation in the codon 347 of the cDNA of part of said gene leads to retinitis pigmentosa disease, can be cited.

Among these target genes wherein mutations cause retinitis pigmentosa and other inherited retinopathies, the RP1 gene can be particularly cited. Indeed, in that RP1 gene, the missense mutation of the active-site Lys-296 in that rhodopsin gene, such as K296E, has been found to produces an opsin with no chromophore binding site and therefore not activated by light, causing autosomal dominant retinitis pigmentosa (ADRP), or a nonsense mutation R677-STOP has also been found to be associated with retinitis pigmentosa in family linked to the RP1 locus (Payne et al., Invest. Opthalmol. Vis. Sci., 41:4069-4073, 2000; Guillonneau et al., Hum. Mol. Genet., 8:1541-1546, 1999; Pierce et al., Nat. Genet., 22: 248-254, 1999; Li et al., Proc. Natl. Acad. Sci. USA, 92: 3551-3555, 1995).

Hypoxia inducible factor-1 (HIF-1) is a transcription factor composed of HIF-1 alpha and HIF-1 beta subunits. HIF-1 transactivates multiple genes whose products play key roles in oxygen homeostasis (Ozaki et al., Invest. Opthalmol. Vis. Sci., 40:182-189, 1999). The gene encoding the transcription factor HIFalpha, for example, which governs the expression of several genes involved in inflammation and neovascularization, can be targeted to cure patients with ocular neovascularization, mainly retinal neovascularization (Wenger, J. Exp. Biol., 203:1253-1263, 2000). Its normal sequence, PCDHG, is conserved in humans (439-464) and in mice (669-693). A chimeroplast (term used in the present specification to also designate a chimeric oligonucleotide) bringing a codon stop can be designed in order to have the expressed protein not be able to promote hypoxia induced neovascularization in human or in mice.

In another aspect, the present invention is directed to a method to treat a disease comprising the administration of an acid nucleic, preferably an oligo- or polynucleotide (DNA or RNA) or chimeric oligonucleotide as defined above, capable of reverting or inducing a mutation in a target gene of target cells, gene expression of which is associated to that disease, in a human or animal host in need of such treatment, wherein the method used for delivering in vivo said nucleic acid into said target cells is the method for delivering in vivo nucleic acid according to the present invention.

In one embodiment, the disease treated by the method of the invention is an inherited pathology, including an inherited retinopathy. Eye diseases that can be treated by the methods of the present invention include, but are not limited to, Bardet-Biedl syndrome, autosomal recessive; chorioretinal atrophy or degeneration, autosomal dominant; cone or cone-rod dystrophy, autosomal dominant; cone or cone-rod dystrophy, autosomal recessive; cone or cone-rod dystrophy, X-linked; congenital stationary night blindness, autosomal dominant; congenital stationary night blindness, autosomal recessive; congenital stationary night blindness, X-linked; Leber congenital amaurosis, autosomal recessive; macular degeneration, autosomal dominant; macular degeneration, autosomal recessive; ocular-retinal developmental disease, autosomal dominant; optic atrophy, autosomal dominant; optic atrophy, autosomal recessive; optic atrophy, X-linked; retinitis pigmentosa, autosomal dominant; retinitis pigmentosa, autosomal recessive; retinitis pigmentosa, X-linked; syndromic/systemic diseases with retinopathy, autosomal dominant; syndromic/systemic diseases with retinopathy, autosomal recessive; syndromic/systemic diseases with retinopathy, X-linked; Usher syndrome, autosomal recessive; other retinopathy, autosomal dominant; other retinopathy, autosomal recessive; other retinopathy, mitochondrial; and other retinopathy, X-linked.

The target genes by disease category are presented in Table 1 below:

TABLE 1

| Disease | Target Genes |
|---|---|
| Bardet-Biedl syndrome, autosomal recessive | ARL6, BBS1, BBS2, BBS4, BBS5, BBS7, MKKS, |
| | PTHB1, TRIM32 and TTC8 gene |
| Chorioretinal atrophy or degeneration, autosomal | RGR and TEAD1 gene |
| dominant | |
| Cone or cone-rod dystrophy, autosomal dominant | AIPL1, CRX, GUCA1A, GUCY2D, RIMS1, SEMA4A and |
| | UNC119 gene |
| Cone or cone-rod dystrophy, autosomal recessive | ABCA4, CNGB3 and RDH5 gene |
| Cone or cone-rod dystrophy, X-linked | RPGR |
| Congenital stationary night blindness, autosomal | GNAT1, PDE6B and RHO gene |
| dominant | |
| Congenital stationary night blindness, autosomal | GRK1, GRM6, RDH5 and SAG gene |
| recessive | |
| Congenital stationary night blindness, X-linked | CACNA1F, NYX and RPGR gene |
| Deafness alone or syndromic, autosomal | WFS1 gene |
| dominant | |
| Deafness alone or syndromic, autosomal | CDH23, MYO7A, PCDH15 and USH1C gene |
| recessive | |
| Leber congenital amaurosis, autosomal dominant | CRX and IMPDH1 gene |
| Leber congenital amaurosis, autosomal recessive | AIPL1, CRB1, CRX, GUCY2D, LRAT, RDH12, RPE65, |
| | RPGRIP1 and TULP1 gene |
| Macular degeneration, autosomal dominant | ARMD1, C1QTNF5, EFEMP1, ELOVL4, FSCN2, |
| | GUCA1B, RDS, TIMP3 and VMD2 gene |
| Macular degeneration, autosomal recessive | ABCA4 gene |
| Macular degeneration, X-linked | RPGR gene |
| Optic atrophy, autosomal dominant | OPA1 gene |
| Optic atrophy, X-linked | TIMM8A gene |
| Retinitis pigmentosa, autosomal dominant | CA4, CRX, FSCN2, GUCA1B, IMPDH1, NRL, PRPF3, |
| | PRPF8, PRPF31, RDS, RHO, ROM1, RP1, RP9 and |
| | SEMA4A gene |
| Retinitis pigmentosa, autosomal recessive | ABCA4, CERKL, CNGA1, CNGB1, CRB1, LRAT, |
| | MERTK, NR2E3, NRL, PDE6A, PDE6B, RGR, RHO, |
| | RLBP1, RP1, RPE65, SAG, TULP1 and USH2A |
| Retinitis pigmentosa, X-linked | RP2 and RPGR gene |
| Syndromic/systemic diseases with retinopathy, | ABCC6, ATXN7, COL11A1, COL2A1, JAG1 and PAX2 |
| autosomal dominant | gene |
| Syndromic/systemic diseases with retinopathy, | ABCC6, ALMS1, CLN3, LRP5, MTP, OPA3, PEX1, |
| autosomal recessive | PEX7, PHYH, PXMP3, TTPA and WFS1 gene |
| Syndromic/systemic diseases with retinopathy, | TIMM8A gene |
| X-linked | |
| Usher syndrome, autosomal recessive | CDH23, MASS1, MYO7A, PCDH15, USH1C, USH1G, |
| | USH2A and USH3A gene |
| Other retinopathy, autosomal dominant | CRB1, FZD4, LRP5, OPN1SW, RB1 and VMD2 gene |
| Other retinopathy, autosomal recessive | CDH3, CNGA3, CNGB3, CYP4V2, GNAT2, LRP5, |
| | NR2E3, OAT, PROML1, R9AP, RBP4, RGS9 and |
| | RLBP1 gene |
| Other retinopathy, mitochondrial | KSS, LHON, MTATP6, MTTH, MTTL1 and MTTS2 gene |
| Other retinopathy, X-linked | CHM, DMD, NDP, OPN1LW, OPN1MW, PGK1 and RS1 |
| | gene |

In a preferred embodiment, the said retinal disease is selected from the group of autosomal dominant, autosomal recessive or X-linked Retinitis pigmentosa.

Among the others eye diseases that can be prevented or treated by the method of the present invention, retinal neovascular diseases can be also particularly cited.

The retinal neovascularization to be treated or inhibited can be caused by diabetic retinopathy, vein occlusion, sickle cell retinopathy, retinopathy of prematurity, retinal detachment, ocular ischemia or trauma. The intravitreal neovascularization to be treated or inhibited can be caused by diabetic retinopathy, vein occlusion, sickle cell retinopathy, retinopathy of prematurity, retinal detachment, ocular ischemia or trauma. The choroidal neovascularization to be treated or inhibited can be caused by retinal or subretinal disorders of age-related macular degeneration, presumed ocular histoplasmosis syndrome, myopic degeneration, angioid streaks or ocular trauma.

In a preferred embodiment, the nucleic acid that is administered is capable of reverting or inducing a mutation of the RP1 gene.

The present invention also comprises a method according to the present invention, wherein the nucleic acid that is administered is capable of reverting or inducing a mutation of the gene encoding the transcription factor HIF1α.

In another aspect, the present invention is directed to a method to obtain an animal model comprising the administration of an acid nucleic, preferably an oligonucleotide or polynucleotide (DNA or RNA) or chimeric oligonucleotide as defined above, capable of reverting or inducing a mutation in a target gene of target cells of that animal, wherein the method used for delivering in vivo said nucleic acid into said target cells is the method for delivering in vivo nucleic acid according to the present invention.

In another aspect, the present invention is directed to a method for the screening of pharmaceutical or cosmetic compounds comprising a step wherein said pharmaceutical or cosmetic compound to be tested is administered to an animal model obtain by the method of the present invention, animal model that has been modified by the administration of an acid nucleic, preferably an oligo- or polynucleotide (DNA or RNA) or a chimeric oligonucleotide as defined above and capable of reverting or inducing a mutation in that target gene, wherein the method used for delivering in vivo said nucleic acid into said target cells is the method for delivering in vivo nucleic acid according to the present invention.

To assist in understanding the present invention, the following examples, which describe the results of a series of experiments, are included. These examples relating to the present invention are illustrative and should not, of course, be constructed as specifically limiting the invention. Moreover, such variations of the invention, now known or later developed, which would be within the purview of one skilled in the art are to be considered to fall within the scope of the present invention hereinafter claimed.

Müller Cells

The integral nature and electrophysiology of Müller cells are described in the review by Bringmann and Reichenbach (Frontiers in Bioscience, 6:77-92, 2001). They describe the structural as well as the functional roles these cells play in order to maintain normal retinal function. In particular, they describe the active maintenance of a negative membrane potential brought about by the efflux of potassium and influx of sodium (by the appropriate ion channels). Due to the inherent membrane potential, it is expected that an applied electric current would have an affect on theses cells, potentially causing activation of these ion channels Cho et al. (FASEB, 13:678-683, 1999; FASEB, 8:771-776, 1994; and references contained within), describe a series of experiments inducing morphological changes upon an application of an AC current, which they propose is due to an electric field induced calcium influx. They also report dc electric field induced microfilament rearrangements that caused structural changes within the cell membrane. Several references within the paper appear to describe a plethora of cell types that undergo morphological changes upon exposure to electric fields.

The Müller Cells span the entire depth of the retina and are in direct contact with the vitreous body. It follows that while the channels are artificially opened and/or compromised, molecules residing in the vitreous, such as oligonucleotides, would passively enter the Müller cells, which by their integral nature, would allow diffusion into the remaining retinal layers.

The integral nature of the Müller Cells would necessitate their involvement in the migration of therapeutics, namely oligonucleotides, from the vitreous to the various layers of the retinal cells.

From the foregoing, it will be appreciated that it would be an advancement in the art to provide a simple and efficient method for ultra-structure change of the Müller Cells to favor the migration of therapeutic nucleic acids into retinal cells, particularly for ocular gene therapy. Nevertheless, there is a need for the development of such new methods that temporarily change the structure of the Müller Cells and that do no alter the photoreceptors.

The inventors have demonstrated that iontophoresis is a simple and efficient method for the temporary elongation of Müller Cells and that this transient elongation facilitates the intraretinal penetration of nucleic acids without altering the photoreceptors. Indeed, eyes analyzed 1 hour after an iontophoretic current application demonstrated enlargement of Müller Cells prolongations with normal integrity of the photoreceptor nuclei. Enlargement of the Müller Cells prolongations were no not detected when the treated eyes were examined 24 hours after iontophoresis. Thus the observed Müller Cells changes induced by the electric current were temporary and that no permanent ultra-structure change was induced by iontophoresis. Particularly, no alteration of the photoreceptors could be detected.

### EXAMPLES

The invention will now be described further in detail with respect to specific embodiments by way of examples, it being understood that these are intended to be illustrative only and the invention is not limited to the materials, amounts, procedures and process parameters, etc. recited therein. All parts and percentages recited are by weight unless otherwise specified.

### Example 1

### Treatment of the Retinal Degeneration of the rd Mouse by Iontopherically Transferring In Vivo a Chimeric Oligonucleotide into Retina Cells

### I Molecular Basis of the Retinal Degeneration in RD Mice

Mice homozygous for the rd mutation display hereditary retinal degeneration and serve as a model for human retinitis pigmentosa. In affected animals, the retinal rod photoreceptor cells begin degenerating at about postnatal day 8 and by four weeks no cones are left. Degeneration is preceded by accumulation of cyclic GMP in the retina and is correlated with deficient activity of the rod cGMP-phosphodiesterase. This enzymatic defect is due to the presence of a nonsense C→A mutation in the rd β-PDE gene. The nonsense mutation creates an ochre stop codon (position 347) within exon 7 and leads to the truncation of the resulting cGMP-phosphodiesterase β-subunit. The absence of a functional cGMP-phosphodiesterase protein in rd/rd mice is responsible for retinal degeneration.

It can be assumed that a reversion of the stop mutation of the rd β-PDE gene will lead to a functional protein in the photoreceptor and the disease is cured. The strategy using chimeric oligonucleotides, proved to be efficient in other models of hereditary diseases due to point mutations, has been chosen for this challenge. So, the chimeraplasty has been used to correct the nonsense mutation responsible for the retinal degeneration of the rd mouse.

The chimeric oligonucleotides were delivered into the targeted tissue using the combination of both, local injection and iontophoresis.

### II Materials and Methods

### Materials

### 1) Chimeric Oligonucleotides

The DNA/2′OMeRNA chimeric oligonucleotides were synthetisized and purified by high pressure liquid chromatography by GensetOligos (France). The oligonucleotides were resuspended in distilled water and quantitated by ultra-violet absorbance at 260 nm. The sequences of the chimeric oligonucleotides are follows:

Specific chimeric oligonucleotide (named Chi) having the following sequence (the 2′OMe RNA nucleotides are underlined):

(SEQ ID NO:1)

5′ CCTTCCAACCTACGTAGCAGAAAGTTTTTACUUUCUGCUACGTAGGU
UGGAAGGGCGCGTTTTCGCGC 3′

Control chimeric oligonucleotide (named Ctr) (the 2′OMe RNA nucleotides are underlined):

(SEQ ID NO:2)

5′ CTACCAAATCCATGGGATTTCCATCAGTTAUUUCUGUCCATCAGGUA
GGAGUGGGCTCGCGTGCGTTC 3′

### 2) Animals

C3H/HeN mice with a nonsense mutation (position 347) were purchased (Iffa Credo). Genotyping to verify the absence or presence of the rd/rd mutation was accomplished by PCR of DNA from tail biopsies and subsequent restriction fragment analysis. The animals were given food and water ad libitum and maintained under pathogen-free conditions of 12 h-light/12 h darkness.

### 3) Coulomb-Controlled Iontophoresis (CCI) System

Iontophoresis was performed using the drug delivery device designed by OPTIS France (as disclosed in U.S. Pat. No. 6,154,671, issued Nov. 18, 2000). A container was designed to allow transcorneoscleral iontophoresis. A platinum electrode was placed at the bottom of the container and two silicone tubes were settled laterally. One tube was used to infuse saline buffer and the other to aspirate bubbles. The CCI electronic unit can delivered up to 2,500 μA for 600 sec. An audio-visual alarm indicated each disruption in the electric circuit ensuring a calibrated and controlled delivery of the product. To proceed with the iontophoresis treatment, the CCI ocular cup was placed on the eye and the other electrode was maintained in contact with the animal.

### Methods

### 1) Injection and Iontophoresis

The experiments were conducted in accordance with the ARVO statement for the use of animals in ophthalmic and vision research. The following treatment was administered on postnatal day (P) 7 and repeated on P9: Mice were anesthetized with an intra-peritoneal injection of chlorpromazine and ketamin. Ocular injections were performed into the vitreous using a glass micro-capillary under microscopic visualization. Just after intravitreal injection, coulomb controlled iontophoresis was performed. The iontophoresis parameters were 300 μA for 300 sec. The negatively charged electrode was placed onto the eye. A solution of phosphate buffered saline (PBS) was continuously pumped into the drug container.

### 2) Oligonucleotide Transfection Analysis Using a Biotinylated Chimeric Oligonucleotide

Biotinylated chimeric oligonucleotide were injected and followed by iontophoresis as described above. The eyes were enucleated 1 h after the treatment, immediately frozen in OCT (Tissue Tek, USA) and sectioned (10 μm). They were fixed in methanol at −20° C. for 10 min. The sections were then washed in 1% Triton X-100 PBS and incubated in a 1/100 streptavidin horseradish-peroxidase PBS solution for 2 h at room-temperature. The sections were washed and the complex was revealed using 3.3° diaminobenzidine tetrahydrochloride in the presence of H2O2. Finally, the sections were counterstained with Hemalun.

3) rd-Mutation Test by Restriction Fragment Analysis of RT-PCR Products

Total RNA was extracted from single retina of rd/rd mice 18 days after the last treatment (P27) by the acid guanidinium thiocyanate-phenol-chloroform method. Retinal total RNA (1 μg) was used as template to synthesize cDNA in a volume of 20 μl using 200U of Moloney leukemia virus (MLV) reverse transcriptase (10 min at 21° C., 1 h at 42° C., 5 min at 55° C., 10 min at 42° C.). Oligonucleotide primers included the sequences 5′-GGC CGG GAA ATT GTC TTC TAC-3′ (SEQ ID NO:3) and 5′-CCC CAG GAA CTG TGT CAG AGA-3′ (SEQ ID NO:4), located at nucleotide positions 921 to 943 and 1258 to 1279 of the β-cGMP-phosphodiesterase cDNA respectively. RT product was amplified by PCR in a volume of 100 μl using 3U of Taq polymerase and primers described above. Thirty PCR cycles were performed in thermal cycler with an initial denaturation of 5 min at 94° C., denaturation temperature of 94° C. for 1 min, annealing temperature of 55° C. for 1 min, extension temperature of 72° C. for 1 min and a final extension of 10 min at 72° C. The PCR buffer contained α-32P dCTP. After each PCR reaction, products were digested with 2.5 units BsaAI and/or 5 units DdeI in the provided buffer at 37° C. overnight, then ethanol precipitated, washed and resuspended in 10 μl gel loading buffer. The products were run on an 8% nondenaturing polyacrylamyde gel at 500 volts for 3 hours. The gel was exposed to a film for 3 days. RNA from +/+retinae and from untreated rd/rd retinae served as controls.

### 4) Immunohistochemistry of Flat-Mounted Retinas

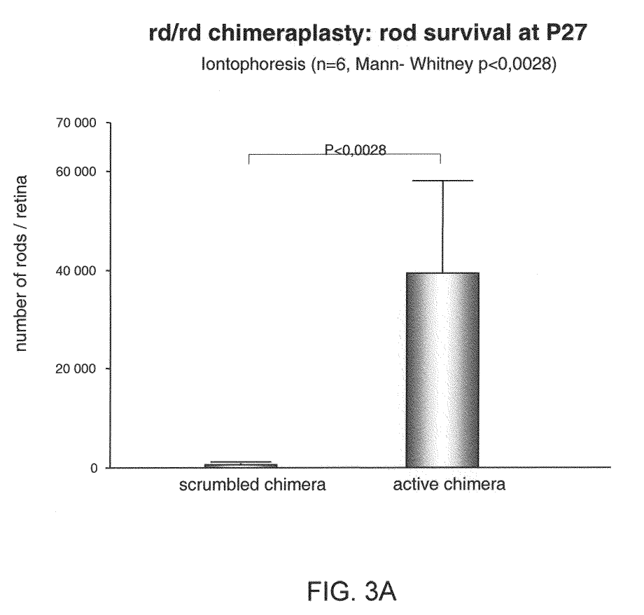

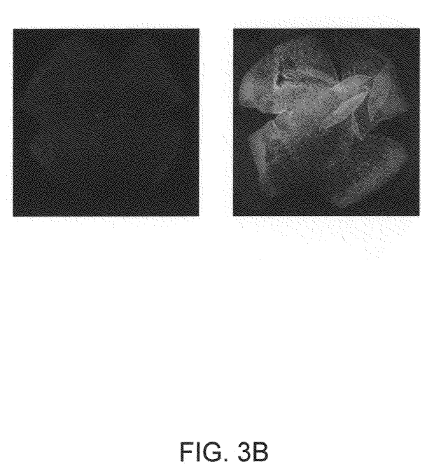

To analyze the survival rate of rod-photoreceptors in control and control treated animals, opsin-immunohistochemistry was performed on whole-mounted retina. The antibody Rho4D2 recognizes specifically opsin, which is the photo-pigment of rod-photoreceteptors. Eyes were enucleated and fixed for 30 min in PBS/Paraformaldehyde 4%. The retinae were dissected and fixed in methanol at −20° C. for 10 min, washed three times in 1% Triton X-100 PBS, incubated over-night in a 1/100 Rho4D2, 1% Triton X-100 PBS solution at room temperature. The retina were then washed, incubated for 2 h at room temperature with an 1/250 anti-mouse Alexa 40 antibody, washed and flat-mounted in glycerol/PBS. They were viewed and photographed by fluorescence microscopy (see FIG. 3B). The photographs were all taken with the same film (Illford 400ASA), exposure time (1 h 30 min), and developed in exactly the same manner. The photographs of flat-mounted retinae were scanned. The number of rod-photoreceptors was measured using a computerized image-analysis system (NIH).

### 5) Statistical Analysis

Results were expressed as mean±standard error of the mean (SEM) (see FIG. 3A). Statistical analyses were performed using the non parametric Man Whitney U test.

### III Results and Discussion

### Chimeraplast Design

Using chimeraplasty rules, a DNA/RNA2′OMe oligonucleotide (named Chi) has been designed, which has the potentialities to revert the C→A point mutation located within codon 347 in the mouse rd β-PDE gene. A control oligonucleotide (named Ctr) contains the same base composition as the active chimeric oligonucleotide but a different sequence.

### Photoreceptor Transfection by Chimeric Oligonucleotides

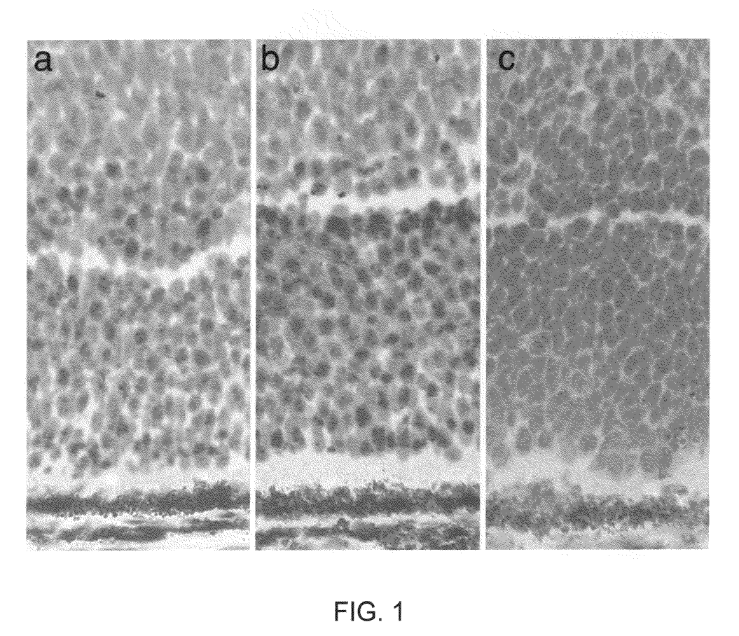

The experiments with the biotinylated oligonucleotide clearly demonstrate that iontophoresis enhances the oligonucleotide uptake in retinal cells, compared to intravitreal injections only as. Notably the uptake in photoreceptors is clearly visible in FIGS. 1A-1C.

Point Mutation Correction Within rd β-PDE mRNA

RT-PCR were performed with rd β-PDE mRNA specific primers on extracted retinae. The rd nonsense point mutation in codon 347 creates a DdeI restriction site and removes a BsaAI site from the wild-type sequence. Digesting the 359 bp β-PDE cDNA with BsaAI or DdeI yields two diagnostic fragments of 120 bp and 239 bp. This method allows the differentiation of the mutated sequence (DdeI sensitive) from the wild-type one (BsaAI sensitive) at the mRNA level.

Intravitreal injection and iontophoresis were performed on postnatal day 7 and 9 mice. RT-PCR experiments followed by restriction digestions were performed in different conditions in order to check the effect of the chimeric oligonucleotide on the rd β-PDE mRNA correction at postnatal day 27.

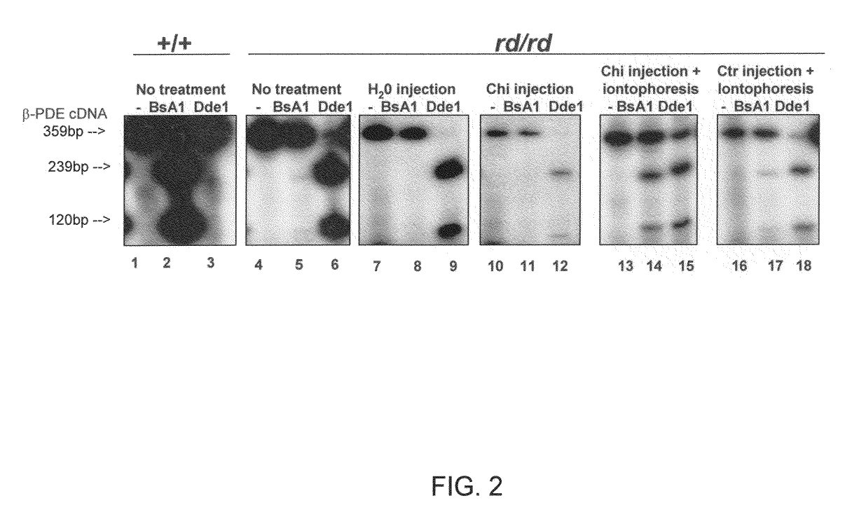

The gel in FIG. 2 shows that:

the rd/rd β-PDE cDNA were totally cut only by DdeI, which recognized only the mutated sequence (lanes 4-6);

the +/+ β-PDE cDNA were totally cut by BsaAI, which recognized only the wild-type sequence (lanes 1-3). Nevertheless, the slight presence of BsaAI digestion product indicated a slight lack of specificity of BsaAI;

the β-PDE cDNA from chimeraplast-treated mice were cut by BsaAI and partly by DdeI only if the intravitreal injection was followed by iontophoresis (lanes 13-15). It demonstrated that the chimeric oligonucleotide Chi could revert the rd point mutation into the wild-type nucleotide. Moreover it indicated that only the combination of both techniques (intravitreal injection and iontophoresis) could allow chimeraplast-mediated gene correction;

the β-PDE cDNA from control chimeraplast-treated mice were cut by DdeI and slightly by BsaAI (lanes 16-18). The BsaAI reactivity could be explained by its lack of specificity already observed after rd/rd β-PDE cDNA digestion (lane 5); and

the β-PDE cDNA from water-treated mice were cut only by DdeI showing that gene correction occurs only in the presence of chimeric oligonucleotides (lanes 7-9).

### Photoreceptor Rescue

The amount of rod-photoreceptors was counted on flat-mounted retina of chimeraplast treated animals and control at P27. In untreated animals and control treated animals, as well as in animals treated with an intravitreal water-injection followed by iontophoresis, the survival at that stage of the disease is negligible. A highly significant increase in rod-photoreceptor-survival can be observed in chimeraplast/iontophoresis treated animals only.

Iontophoresis is known to be a non-invasive process to deliver drugs using a low-intensity current. It uses an electrode of the same polarity as the charge on the drug to drive ionic drugs into the tissues. The present inventors have demonstrated that iontophoresis can be used to enhance the nucleic acid penetration into cells tissue, such as chimeric oligonucleotide DNA/2′OMeRNA type, particularly into ocular cells after intra- or peri-ocular injection and to enhance retinal transfer or penetration after or before or simultaneously to intraocular injection.

### Example 2

### Combination of Oligonucleotides (ODNs), Intravitreous Injection, and Saline Transpalpebral Iontophoresis on the Delivery of ODNs to Photoreceptors in the New Born rd1/rd1 Mice

### Introduction

Direct iontophoresis enhances the intraocular levels of locally applied drugs both in experimental models and in patients. Different types of devices have been designed to apply the current on the cornea, the sclera or both, with drug application on the eye surface in containers of various forms and materials. This type of iontophoresis procedure can be qualified as “direct ocular iontophoresis”. Direct ocular iontophoresis has also been used to enhance the intra-tissue and intra-cellular penetration of oligonucleotides (ODNs). The mechanisms of drug penetration facilitation by iontophoresis include electrorepulsion, electroosmosis and current-induced tissue permeation. Post-iontophoretic transport of drugs has been described in the skin and results from tissue changes that may persist for a limited period of time after current application. To study the penetration of ODNs into photoreceptor cells of new born rd1/rd1 mice eyes, another procedure that associates electric current application at the eye surface using a saline transpalpebral iontophoresis with intravitreous injection of ODNs was evaluated. Various conditions of iontophoresis (anodal versus cathodal, current intensity and, time between injection and current application) were evaluated.

### Materials and Methods

### Animals

C3H/HeN mice homozygous for the nonsense mutation (amino acid position 347) in the β-PDE gene (Janvier, Le Genest, France) were used (36 mice, 72 eyes). Mice were maintained in clear plastic cages and subjected to a standard light: dark cycle of 12 hours. Experiments were conducted in accordance with the ARVO Statement for the Use of Animals in Opthalmologic and Vision Research and the institutional guidelines regarding animal experimentation in Ophthalmic and Vision Research.

### Oligonucleotide (ODN)

ODN was synthesized and purified by high pressure liquid chromatography by Proligo (Paris, France). A 25 mers phosphorothioate ODN, encoding for the sense wild-type β-PDE gene sequence (25-6x6PS-WTS, 5′-CsCsTsTsCsCsAACCTACGTAGCAsGsAsAsAsGsT-3′; SEQ ID NO:5) and 5′ labeled with Hex, was used for histological evaluation of ODN distribution.

### Iontophoresis and Intravitreous Injection

Eyelids of PN7 rd1/rd1 mice were opened with a scalpel (Swann Morton, Peynier, France) under topical tetracaine 1% drops anesthesia (Novartis Ophthalmics SA, Rueil Malmaison, France).

Transpalpebral iontophoresis system (patent # FR2830766) was used. Eye-glass-shaped aluminum foil and disposable medical grade hydrophilic polyurethane sponge (3.2 mm thick, 1.5×0.7 cm length by width, Optis, Levallois, France), were soaked in PBS (phosphate buffered saline: 0.2 g/L KCl, 0.2 g/L KH2PO4, 8 g/L NaCl, 2.16 g/L Na2HPO4 7H2O, pH7.4) and used as the active electrode (FIG. 1C). The electrode covered both closed eyelids of the treated newborn mouse. The return electrode was connected to the neck of the mouse. An audio-visual alarm indicated any disruption of the electric circuit ensuring a controlled delivery of the current.

Intraocular injections were carried out with ES TransferTips microcapillaries (Leica, Rueil Malmaison, France) and cut at 2 mm from their extremity, leading to a 60 μm injecting hole. Microcapillaries were linked to a Micro4™ microsyringe pump controller (World Precision instruments, Sarasota, USA). One μL of ODN (500 μM) was injected into the vitreous at a constant pressure of 200 mL/sec. The position of the needle was monitored by observation with a dissecting microscope through a glass cover slip placed on the corneal surface. To limit loss of the injected solution and allow the intraocular pressure to equilibrate (as observed by the return of normal iris perfusion), the micropipette needle was left in place for 10 sec after the injection before withdrawal.

### Treatment Protocols

The ODN distribution was evaluated at one hour after the end of the procedure. Anodal or cathodal transpalpebral saline iontophoresis (respectively positive or negative electrode connected to the eyelids) was performed with a current of 1.5 mA for 5 min (1.43 mA/cm2) before the intravitreous injection of ODN (4 eyes for each experiment). Cathodal iontophoresis was then tested when applied immediately after the intravitreous injection of ODNs (4 eyes). The results derived from this initial set of experiments showed that cathodal iontophoresis performed prior to the intravitreal injection led to the highest ODN penetration in the ONL. This condition was therefore used to evaluate the effect of lower current intensity (0.5 mA for 5 min) or higher current intensity (2.5 mA for 5 min), as well as the duration of current-induced permeation by injecting ODN at various time point after iontophoresis (acute, 1, 3 and 6 hours) (4 eyes for each experiment). With the optimal conditions defined, the kinetics of fluorescent ODN distribution was evaluated at 1, 6 and 24 hours after treatment (4 eyes for each condition). Control animals received intravitreous injection of 1 μL Hex (500 mM) (Invitrogen, Cergy Pontoise, France) or PBS with or without previous cathodal iontophoresis (4 eyes for each condition). Four additional eyes were treated and sacrificed at one hour to evaluate the integrity of the injected ODN by acrylamide gel electrophoresis.

Mice were sacrificed by an intraperitoneal lethal dose of pentobarbital (6 g/100 ml; Ceva Santé Animale, Libourne, France). The eyes were enucleated and processed for the various tests as described below.

### Evaluation and Quantification of Fluorescent ODN Distribution

At various time points after treatment, the eyes were enucleated, rinsed in PBS and embedded with Tissue-Tek OCT-compound (optimal cryotechnique compound, Bayer Diagnostics, Puteaux, France) for cryo-sectioning. Sections (10 μm) were fixed in 4% paraformaldehyde (Merck Eurolab, Strasbourg, France) for 5 min at room temperature, washed in PBS, counter-stained for 2 min with DAPI (4′,6-diamino-2-phenylindole) (1/3000) (Sigma-Aldrich, Saint-Quentin Fallavier, France), washed in PBS, mounted in Gel Mount (Microm Microtech, Francheville, France). For localization of labeled ODN, sections were examined under a fluorescent microscope (Aristoplan, Leica) and images were captured using a digital SPOT camera (Optilas, Evry, France) with a constant exposure time of 3 seconds (for 2.5 times magnification) or 0.8 seconds (for 25 times magnification).

For fluorescence quantification, for each eye, 5 sagital sections through the entire eye were photographed (3 pictures per section, n=15 values for each eye). All pictures were taken with a 25× objective and a similar exposure time of 0.8 seconds. Intensity of the outer nuclear layer (ONL) fluorescence was quantified by using the luminosity feature of Photoshop. Tissue and background regions were manually selected. Mean pixel brightness was determined for each region by using the “Histogram” imaging feature. To control for differences in background levels among images, the mean brightness level per pixel of the tissue region was divided by the background region from each section image.

### ODN Integrity

Neural retinas were dissected one hour after intravitreous injection of Hex-labeled ODNs with prior cathodal saline iontophoresis (1.5 mA for 5 min) (n=4). Pools of two retinas were placed in 500 μL of digestion buffer (50 mM Tris pH 8, 10 mM EDTA pH 8, 0.5% SDS) containing 0.5 mg/ml of proteinase K and incubated for 90 min at 56° C. After incubation, the DNA samples were extracted once with phenol (1/1; v/v) and once with chloroform/isoamylalcohol (1/1; v/v; 24/1; v/v). The samples were then extracted once with isobutanol (1/2; v/v) and once with diethyl ether (1/1; v/v), precipitated with ethanol/ammonium acetate (1/2/1/3, v/v/v), dried, re-suspended in 20 μl TE buffer (10 mM Tris, pH 8, 1 mM EDTA, pH 8) and treated with DNAse1. Ten μl of each sample was denatured with 2.5 μl formamide (90% formamide, 0.05% bromophenol blue) and then run for electrophoresis on 12% denaturating polyacrylamide gel and fluorescent Hex ODNs were visualized and photographed (Typhoon, Amersham).

### Structure Analysis of the Retina

For structure analysis of the whole ocular globe, eyes treated with cathodal 1.5 mA iontophoresis for 5 minutes prior to intravitreal injection were studied. Mice were sacrificed 1, 6, and 24 hours after the intravitreous injection, the globes enucleated, fixed and studied (4 eyes for each condition). Cryosections at the optic nerve level were counter-stained with hematoxylin and eosin, examined using a photonic Aristoplan microscope (Leica, Rueil Malmaison, France) and photographed using a digital SPOT camera (Optilas). To assess potential tissue changes associated with the treatment procedure, transmission electron microscopy (TEM) was performed. For this purpose, 4 eyes received iontophoresis followed by ODN injection, 4 eyes received ODN injection without iontophoresis and 4 other eyes received iontophoresis alone. At one and 24 hours after treatment (2 eyes per time point) the mice were sacrificed, the eyes enucleated and fixed in 2.5% glutaraldehyde of cacodylate buffer (Na 0.1 M, pH7.4). After 1 hour, the globes were dissected at the limbus, the posterior eye ball post fixed for 3 hours and cut in 4 parts. Tissues were post-fixed in 1% osmium tetraoxyde in cacodylate buffer (Na 0.1M, pH7.4) and dehydrated in graduated ethanol solution (50, 70, 95, 100%). The tissues were then included in epoxy resin and oriented. Semi-thin sections (1 μm), obtained with an ultramicrotome Reichert Ultracut E (Leica), were stained by toluidin blue. Ultra-thin sections (80 nm) were contrasted by uranyl acetate and lead citrate and observed with an electron microscope JEOL 100CX (JEOL, Tokyo, Japan) under 80 kV.

### Statistics

Results were expressed as means±SD and compared using the analysis of variance (ANOVA) test with post Fisher test. P<0.05 was considered as significant.

### Results

Enhanced ODN Delivery to Retinal Cells of rd1/rd1 Mice

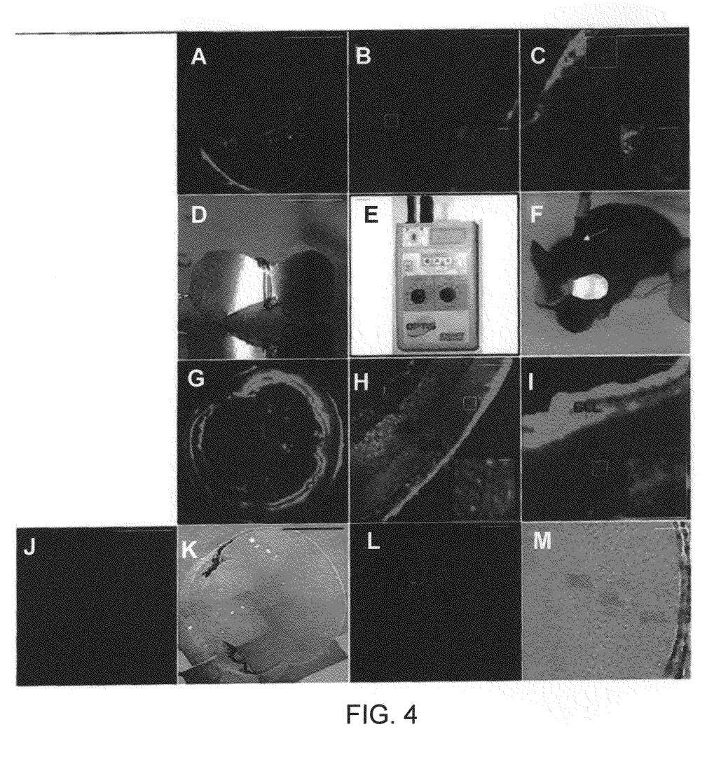

One hour after the intravitreal injection of Hex-labeled WTS ODN in rd1/rd1 PN7 mice, without applied current, fluorescence was observed in the nuclei of the ganglion cell layer (GCL) and in the most superficial nuclei of the inner nuclear layer (INL) (FIGS. 4A, 4a and inset in 4a). No fluorescence was detected in the outer nuclear layer (ONL) (FIGS. 4A and 4a). One hour after intravitreous injection of the fluorochrome (Hex) alone, diffuse fluorescence was observed in the vitreous and in cells of the inner nuclear layer but without specific accumulation in the cell nuclei (FIG. 4B, inset).

When saline iontophoresis (FIGS. 4C, 4D and 4E) was applied and immediately followed by injection of the labeled ODN, intense fluorescence was observed in all retinal layers (FIGS. 4F and 4f). Specific localization to the retinal cell nuclei was seen in these cases (inset FIG. 4f). When saline iontophoresis was applied and followed immediately by the injection of the fluorochrome (Hex) alone (without ODN), diffuse fluorescence was observed in all retinal layers (FIG. 4G). However, this fluorescence was not accumulating specifically in the cell nuclei (inset in FIG. 4G). No fluorescence was observed in the retinal layers of PBS-treated eyes (FIGS. 4H and 4h) or non-injected control eyes (with or without saline iontophoresis) (data not shown) (4 eyes for each condition).

ODN integrity was confirmed by acrylamide gel electrophoresis of the DNA extracted from retinas of treated eyes one hour after iontophoresis followed by injection (data not shown).

### Effect of Iontophoretic Parameters on ODN Penetration

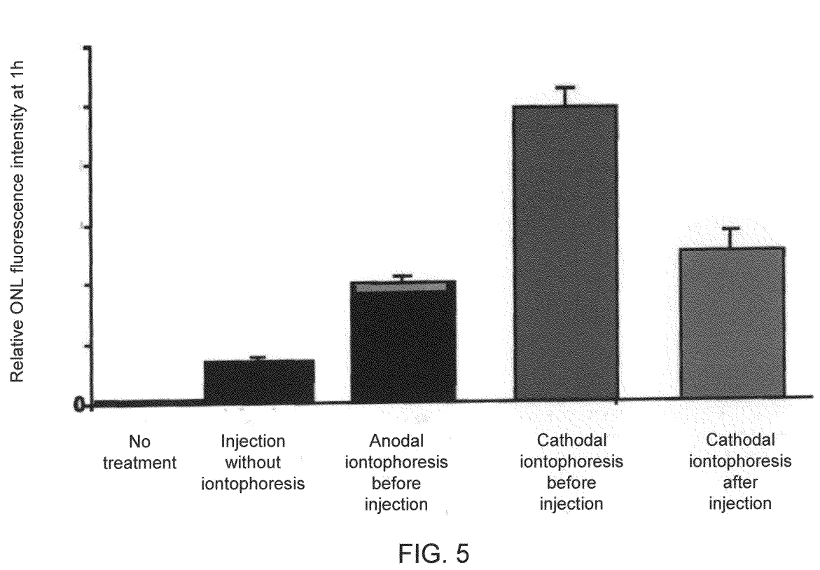

Iontophoresis was performed prior to ODN injection in order to limit the risk of infection and of potential reflux of ODNs from the globe by mechanical pressure of the probe on the eyelids. Cathodal or anodal iontophoresis (1.5 mA for 5 min) prior the intravitreous injection of ODNs showed that the application of current enhanced ODN penetration when compared to injection without iontophoresis (P<0.05) or no treatment (P<0.05) (FIG. 5: * and **). Furthermore, prior cathodal saline iontophoresis significantly enhanced ODN penetration in the ONL cells when compared to anodal saline iontophoresis (P<0.05: **). Performing cathodal saline iontophoresis immediately prior to the intravitreous ODN injection significantly enhanced the ODN penetration in ONL when compared to application of cathodal iontophoresis immediately following ODN injection (P<0.05:**). From all tested conditions, cathodal iontophoresis performed immediately prior to ODN injection yielded the highest ODN penetration in the ONL of treated mice eyes. The later condition was therefore used to evaluate further parameters.

### Effect of Current Intensity

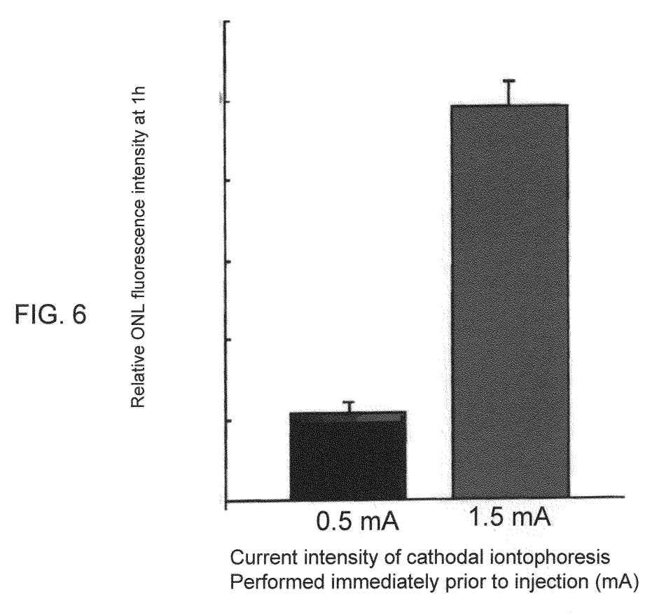

Iontophoresis with 0.5 mA significantly decreased the ODN penetration in ONL when compared to 1.5 mA (P<0.05:*) showing that ODN penetration in the ONL depends on the amount of applied current (FIG. 6). Increasing the current intensity to 2.5 mA (for 5 minutes) induced electric skin burns and pain (data not shown). This condition was therefore not further studied.

### Duration of Post-Iontophoretic Facilitation of ODN Penetration

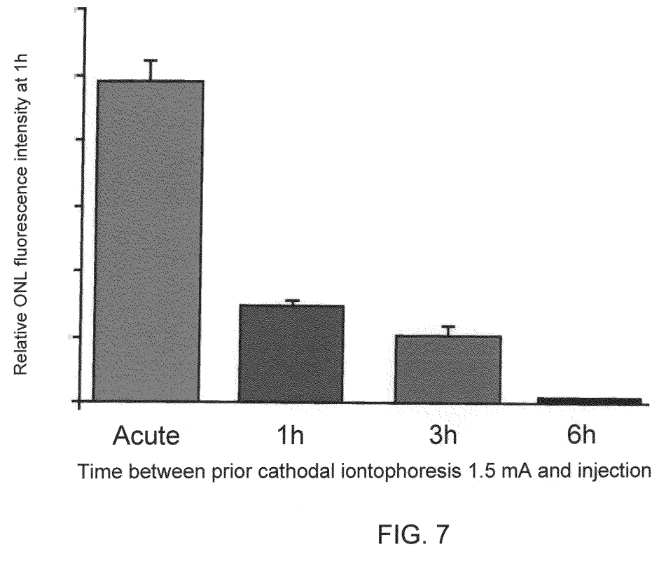

The post-iontophoretic enhancement of ODN penetration decreased with the increased interval between iontophoresis and injection. Facilitation of ODN penetration in the ONL was highest with the shortest interval, during at least 3 hours, and vanished when ODN injection was performed 6 hours after saline iontophoresis (FIG. 7). These results demonstrate that the iontophoresis facilitation of intraretinal penetration is temporary.

### Kinetics of ODN Distribution in the Retina

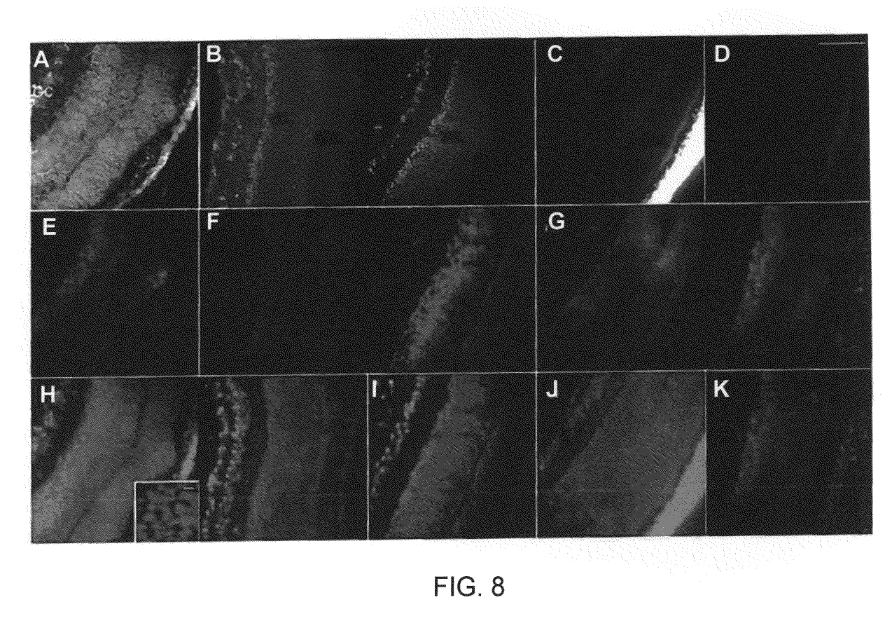

Using the optimal treatment parameters (cathodal iontophoresis 1.5 mA for 5 min prior to intravitreous injection), most intense ONL fluorescence was observed 1 hour after the ODN injection decreasing rapidly at later time points (FIG. 8). Indeed, already at 4 and 6 hours after treatment, it was noted that fluorescence was decreasing in the outer retina (FIGS. 8B and 8C). At 8 hours, ODN fluorescence remained only in the ganglion cell layer (FIG. 8D), and no fluorescence is observed in the neurotina at 24 hours (FIG. 8E). This kinetic suggests that Hex-labeled ODNs may be rapidly degraded after their intracellular penetration.

### Light and Electron Microscopy Observations

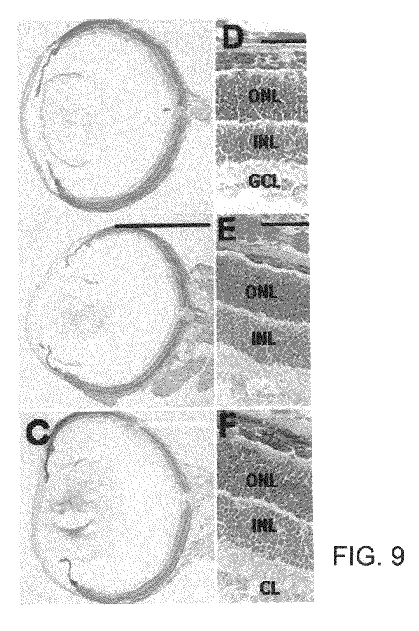

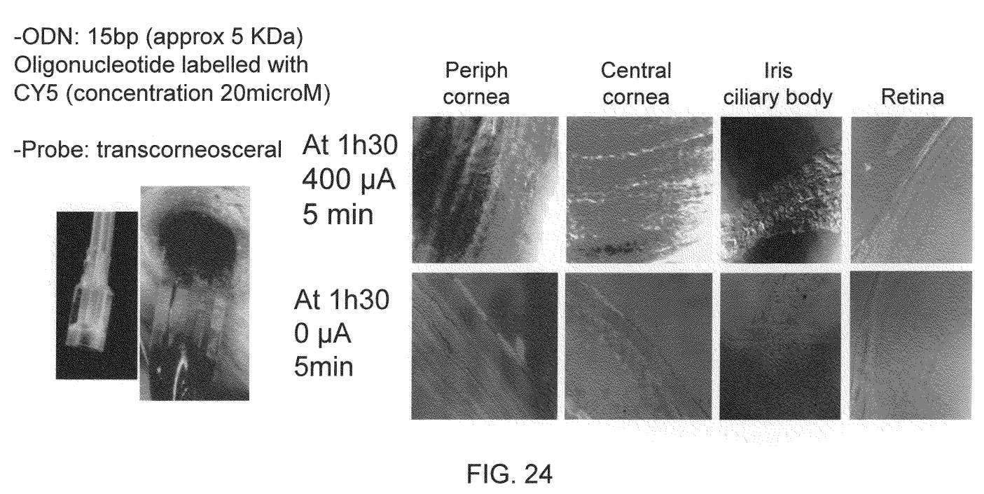

The ocular gross histology structure was not affected by the iontophoresis application. No lesion or cell damage was detected at 1 hour (FIGS. 9A and 9a), 6 hours (FIGS. 9B and 9b) or 24 hours (FIGS. 9C and 9c) after treatment.

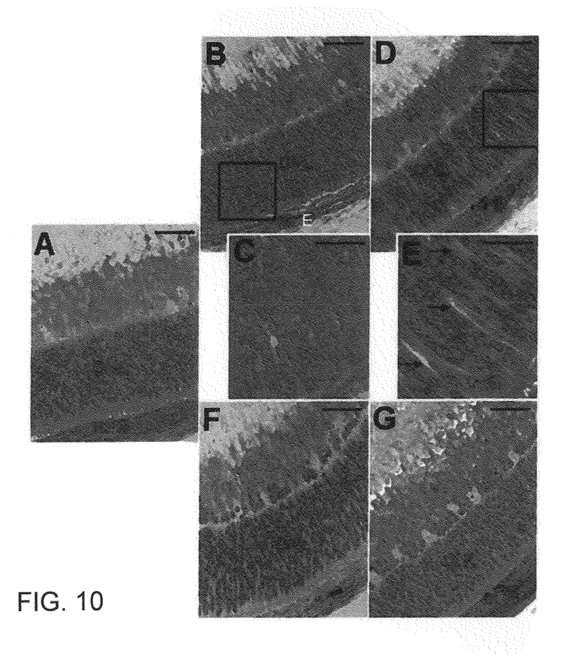

Analysis of semi-thin sections revealed that one hour after application of saline iontophoresis inter nuclei spaces within the INL and ONL were increased. Linear enlargements can be followed from the outer rows of the INL up to the external limiting membrane. This localization is suggestive of Müller glial cell (RMG) prolongations. Note that nuclei in the INL and the ONL have normal structure and do not show any sign of apoptosis or necrosis (FIGS. 10C and 10c). No such changes were observed in untreated control retinas or in eyes without current application (FIGS. 10A, 10B and 10b). Twenty four hours after iontophoresis application, internuclear spacing was no more observed and the ONL has regained a normal architecture (FIG. 1E).

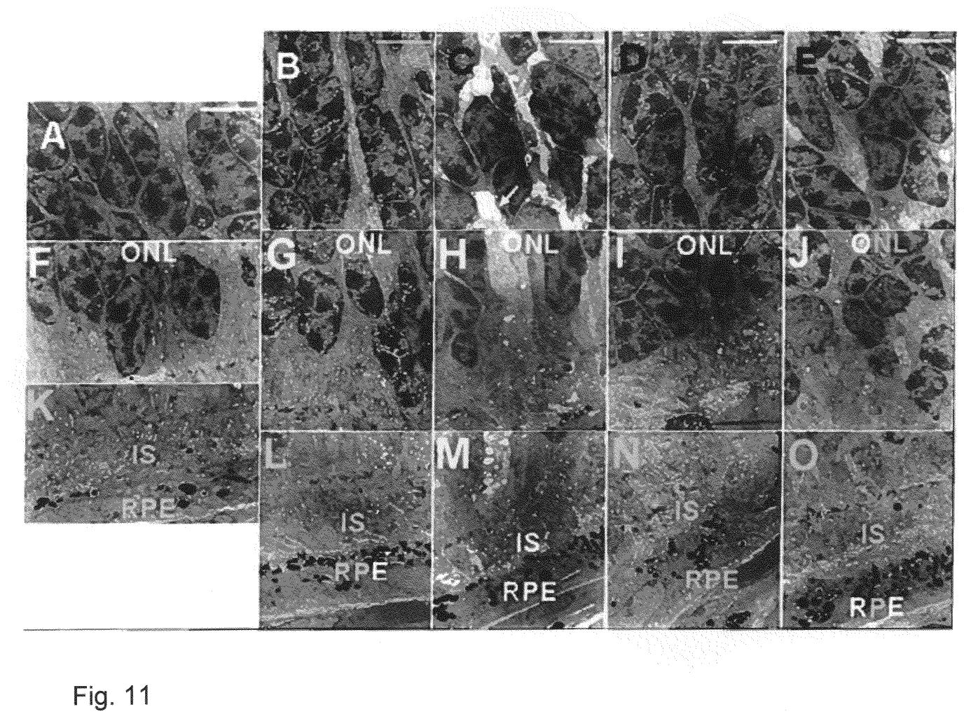

TEM analysis showed that in eyes receiving injection without electric current applied or in untreated control eyes, the retinas retained a normal structure without any detectable changes (FIGS. 11A and 11B). Eyes analyzed 1 hour after iontophoretic current application demonstrated enlargement of RMG prolongations (FIG. 11C, arrow) with normal integrity of the photoreceptor nuclei. Enlargement of the RMG prolongations were no more detected when the treated eyes were examined 24 hours after iontophoresis (FIG. 11E). Thus, the observed RMG changes induced by the electric current were temporary and that no permanent ultra-structure change was induced by the optimal iontophoresis parameters used in this study. Particularly, no alteration of photoreceptors could be detected.

### Discussion

The results show that the application of transpalpebral saline iontophoresis enhances ODN penetration into photoreceptors. The facilitation and enhancement of penetration were associated with the intensity of the current and from this study a 1.5 mA (1.43 mA/cm2) current applied for 5 minutes was determined to be efficient and safe for new born mice eyes. No structural damage of the treated eyes has been observed and the normal architecture of the retina was preserved using these iontophoresis parameters in newborn (PN7) mice. For direct ocular iontophoresis, the active electrode is in contact with the drug solution and electrorepulsion is thought to facilitate the penetration of drug in ocular tissues. However, electrorepulsion is responsible only for a part of the current effect, as facilitated diffusion of non-charged molecules can also be achieved. Electroosmosis is another mechanism of drug penetration acting through a flow process (vol/distance/time). Electroosmosis-induced drug penetration is particularly important for larger molecules. Increased “passive” permeability for a limited period of time after the application of current was also observed in the skin. In these experiments, it was demonstrated that the slow recovery of skin impedance following iontophoresis was due to the movement of ions in response to electric field and that the resulting post-iontophoretic enhanced-diffusion was not associated with damage to the skin barrier. Application of saline iontophoresis before the local instillation of phenylephrine increased the observed vasoconstriction effect of this drug. Most of the studies elucidating the different mechanisms of iontophoresis drug penetration and facilitation have been conducted on the skin. The results obtained by the present study show that saline iontophoresis also influences the permeability of intraocular tissues to charged molecules such as ODNs. Iontophoresis prior to intravitreous injection is more efficient than if the current is applied after injection. One hypothesis is that when iontophoresis is applied after intravitreous injection, potential extraocular diffusion of the injected ODNs due to the mechanical pressure may be responsible for this observed phenomenon.

The ODNs used in this study were negatively charged and with a molecular weight of 7591 g/mol. These characteristics can allow them to penetrate through the internal limiting membrane. However, the exact mechanisms responsible for the transport from the inner retina to the photoreceptor cells remain ill understood. Such transport is probably highly regulated and does not follow passive diffusion. Simple direct vitreous injection of ODNs does not lead to their penetration into photoreceptors. On the other hand, when saline iontophoresis is performed with the intravitreous injection of ODNs, penetration in the photoreceptors cell nuclei is observed. The only changes observed in the retina using semi-thin histology were enlarged linear spaces observed from the outer row of the INL up to the extern limiting membrane at one hour after saline iontophoresis. The localization of these spacing may indicate that changes have occurred in RMG prolongations. Interestingly, this phenomenon was reversible since it could not be observed at 24 hours after the application of the current. One hypothesis may be that RMG cells could participate to the increased penetration of ODN in the photoreceptors. Increased transport vacuoles were observed in the corneal epithelium when direct constant current iontophoresis was applied. This effect was temporary and lasted only for a few hours. In the present study, reversal to normal retinal ultra-structure (without persistent enlargement of RMG elongations) was observed 24 hours after the current application.

In conclusion, saline iontophoresis prior to intravitreous injection of ODNs was demonstrated to facilitate the migration of these charged molecules and enhances their penetration into the retina photoreceptors. Post-iontophoretic enhancement of charged molecules is therefore demonstrated in the retina. Whether prior saline iontophoresis can also facilitates the intra-retinal penetration of larger size ODN molecules or plasmids remains to be explored.

### Example 3

### Single-Stranded Oligonucleotide Mediated In Vivo Gene Repair in the rd1/rd1 Retina

The purpose of this study was to test iontophoresis enhanced the penetration of oligonucleotide in the photoreceptors nuclei and induce targeted gene repair of a point mutation in vivo following iontophoretic delivery of the oligonucleotide.

The rd/rd1 mouse is a model of rapid retinal degeneration. It results in part from a point mutation in the gene encoding the β-subunit of rod receptor cGMP-phosphodiesterase (β-PDE), leading to a stop codon (Tyr347Ter) and subsequent truncated protein. In rd/rd1 mouse, rod photoreceptor loss is complete by postnatal day (PN)21. Mutations in the same gene are responsible for retinal degeneration in patients with retinitis pigmentosa.

Targeted gene repair is a non-viral gene therapy strategy, which aims at correcting mutations in genomic DNA by using RNA/DNA oligonucleotides (RDOs) or single-stranded DNA oligonucleotides (ssODNs). This gene therapy strategy should allow for a permanent correction of the genomic DNA and for normal physiologic regulation of the corrected gene by its endogenous promoter. Targeted gene repair has been effective in inducing genotypic and phenotypic corrections both in vitro and in several animal models of various disorders such as hemophilia, Crigier-Najjar syndrome type 1, albinism, Duchenne muscular dystrophy, hyperlipidemia type 2, and sickle cell disease. The majority of in vivo studies have used RDOs. Recently, successful repair has been described in vivo with phosphorothioate ssODNs. ssODNs present advantages when compared to RDOs. Their synthesis is easier and less expensive, and they are more stable. Moreover the induced repair is more reproducible. Reproducibility being one of the major limitations of gene repair using RDOs, enhanced reproducibility with ssODNs may be of the utmost interest.

Previous attempts at gene correction in the rd1/rd1 mice in vivo using intravitreal or subretinal injections of ODNs were inconclusive, possibly due to inefficient DNA delivery to photoreceptor nuclei (Stodulkova E. et al. IOVS, 42:ARVO Abstract 1873, 2001). Within the eye, transfection of photoreceptors is one of the crucial issues in retinal gene therapy and injected ODNs might not efficiently target photoreceptor cells. It is demonstrated that rd1 mutation can be corrected by gene repair in different non ocular cell lines using LNAs and phosphorothioate ODNs designed to correct the point mutation in the β-PDE gene. To transpose these results to the rd1/rd1 mice in vivo, the effect of current to enhance the photoreceptor delivery of ODN in the retina is herein evaluated. Low current density iontophoresis indeed safely promotes intraocular penetration of drugs and gene fragments. It has been previously observed that iontophoresis enhances the intracellular penetration of intact ODNs in corneal cells. It has been evaluated whether iontophoresis performed immediately before the intravitreal injection of ODNs enhances their penetration into mouse photoreceptor nuclei. In vivo results using this iontophoresis procedure to deliver phosphorothioate and LNAs ODNs in the rd1/rd1 mice retina have shown beneficial effects on photoreceptors survival. It is shown herein that optimized delivery of specific phosphorothioate ODNs designed to correct the point mutation in the β-PDE gene, using iontophoresis, induces genotypic and phenotypic changes of the rd1/rd1 retina. Conversion of the mutant base to wild-type in this model is associated with appearance of β-PDE immunoreactivity in retinal cells, partial preservation of rhodopsin immunoreactivity, and increased photoreceptor cell counts. The genotypic and phenotypic data indicate that limited targeted gene repair was achieved in vivo in the rd1/rd1 neural retina.

### Methods

### Animals

C3H/HeN mice homozygous for the nonsense mutation (amino acid position 347) in the β-PDE gene (Janvier, Le Genest, France) were used. Wild-type mice C57B16/Sev129 served as positive controls. Mice were maintained in clear plastic cages and subjected to a standard light: dark cycle of 12 hours. Experiments were conducted in accordance with the ARVO Statement for the Use of Animals in Opthalmologic and Vision Research and the institutional guidelines regarding animal experimentation in Ophthalmic and Vision Research.

### Oligonucleotides

ODNs used were synthesized and purified by high pressure liquid chromatography by Proligo (Paris, France). ODNs in distilled water were quantified by absorbance at 260 nm. Sense (S) and antisense (AS) wild-type allele of the β-PDE gene sequence were assayed [WTS (5′-C*C*T*T*C*C*AACCTACGTAGCA*G*A*A*A*G*T-3′ SEQ ID NO:6), WTAS (5′-A*C*T*T*T*C*TGCTACGTAGGTT*G*G*A*A*G*G-3′ SEQ ID NO:7)] as well as scrambled ODNs [WTSscr7 (5′-C*C*T*T*C*C*AACAACGTCTGCA*G*A*A*A*G*T-3′ SEQ ID NO:8), WTSscr25 (5′-A*A*T*C*A*C*AGTTGCCTATAGG*A*C*C*C*C*A-3′ SEQ ID NO:9)]. (Nomenclature: WTS/AS=wild type sense/antisense, 25=25 mers, 6x6PS=six phosphorothioate linkages at each 5′ and 3′ positions, *=phosphorothioate linkage, scr7/25=scrambled sequence of the 7 central bases/the entire 25 mers ODN). The single strand ODN WTS, used for histological evaluation of ODNs distribution, was 5′ labeled with CY3.

### Iontophoresis and Injection

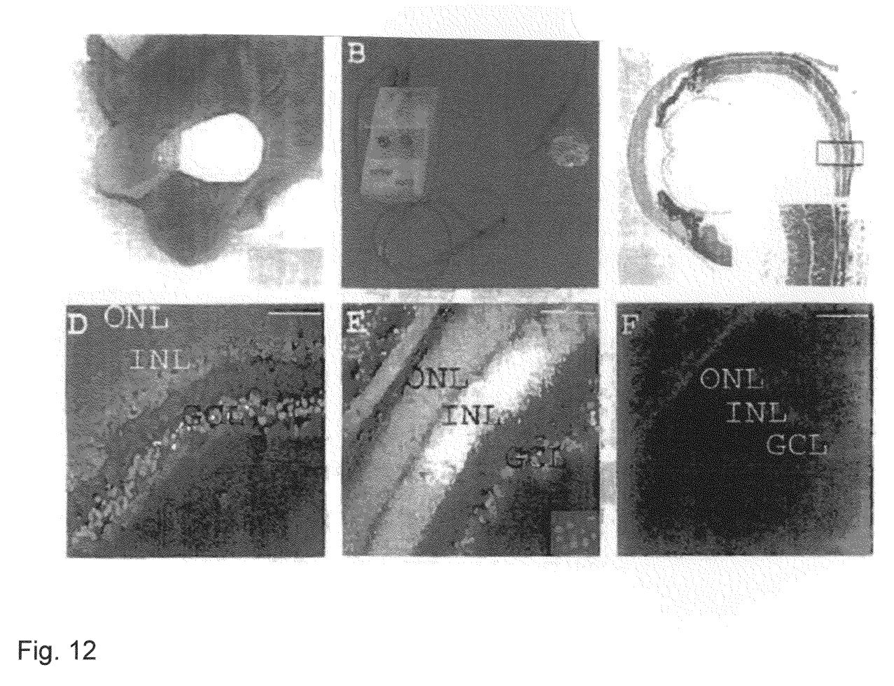

Transpalpebral (across closed eyelids) iontophoresis system (patent # FR2830766) vas used. It was found that applying transpalpebral iontophoresis immediately after or before intravitreal injection of ODNs leads to the same penetration efficiencies. Therefore, iontophoresis was performed immediately prior to the intravitreal injection of ODNs to avoid manipulation of the injected pups' eyes and reduce the potential danger of secondary infection. Prior to iontophoresis, tetracaine 1% drops (Novartis Ophthalmics SA, Rueil Malmaison, France) were instilled. Eye-glass-shaped aluminum foil and disposable medical grade hydrophilic polyurethane sponge (3.2 mm thick, 1.5×0.7 cm length by width, Optis, Levallois, France), were soaked in PBS (phosphate buffered saline: 0.2 g/L KCl, 0.2 g/L KH2PO4, 8 g/L NaCl, 2.16 g/L Na2HPO4 7H2O, pH 7.4) and used as the active electrode (FIG. 12A). The electrode covered both closed eyelids of the treated newborn mouse. The return electrode was connected b the tail and hind foot pads of the mouse. Anionic iontophoresis (negative electrode connected to the eyelids) was performed with a 1.5 mA current for 5 min (1.43 mA/cm2 (FIG. 12B). An audio-visual alarm indicated any disruption of the electric circuit ensuring a controlled delivery of the current.

The pup's eyelids were then opened with a scalpel (Swann Morton, Peynier, France) and intraocular injections were carried out with borosilicate micropipette needles (Phymep, Paris, France) pulled with a pipette puller (model 720, Kops Instrument, Tujunga, Calif., USA) and cut at 2 mm from their extremity, leading to a 60 μm injecting hole. Micropipette needles were linked to an Eppendorf microinjector 5242 (Roucaire, Velizy, France). One μL of PBS or ODN (concentrations stated below and in figure legends) was injected into the vitreous. The position of the needle was monitored by observation under a dissecting microscope through a glass cover slip placed on the corneal surface. To limit loss of the injected solution and allow the intraocular pressure to equilibrate (as observed by the return of normal iris perfusion), the micropipette needle was left in place for 10 sec before withdrawal.

For tissue harvest, mice were sacrificed by a lethal dose of pentobarbital (6 g/10 mL; Ceva Santé Animale, Libourne, France) injected intraperitoneally.

### Localization of ODN

Eight eyes of rd1/rd1 mice at PN7 underwent a single transpalpebral iontophoresis (anionic, 5 min, 1.5 mA) followed by an intravitreal injection of 1 μL of CY3-labeled WTS ODN (1 μL of 272 μM). For the control groups, rd1/rd1 mice al PN7 received either iontophoresis followed by PBS injection, ODN injection without iontophoresis, iontophoresis without injection, or had no treatment (8 eyes for each condition). Animals were sacrificed one hour after treatment. The eyes were enucleated, rinsed in PBS, and embedded with Tissue-Tek OCT compound (Bayer Diagnostics, Puteaux, France) for cryo-sectioning. Sections (10 μm) were fixed in 4% paraformaldehyde (Merck Eurolab, Strasbourg, France) for 5 min at room temperature, washed in PBS, counter-stained for 2 min with DAPI (4′,6-diamino-2-phenylindole) (1/3000 dilution) (Sigma-Aldrich, Saint-Quentin Fallavier, France), washed in PBS, mounted in Gel Mount (Microm Microtech, Francheville, France) and examined under a fluorescence microscope (Aristoplan, Leica, Rueil Malmaison, France) with HBO103w lamp and a digital SPOT camera (Optilas, Evry, France). For each eye, sections at the optic nerve level were counter-stained with hematoxylin and eosin for structural analysis.

### Choice of Optimal ODN and Dosing Based on Phenotypic Changes

### ONL Cell Counting

At days PN4, 6, and 8, rd1/rd1 mice received saline iontophoresis prior to PBS or ODN (1 μL of 500 μM) intravitreal injections. The choice of optimal ODN type to be used for all further experiments was based on quantification of photoreceptor survival at PN28, as determined by direct ONL counting on retina sections.

Eyes of PN28 untreated (6 eyes), PBS-(6 eyes), or WTAS ODN- and WTS treated (8 eyes for each) rd1/rd1 mice were enuclealed, quickly frozen in Tissue-Tek OCT-compound (Bayer Diagnostics), and sectioned (10 μm). For each eye, five sections that included optic nerve were stained with hematoxylin and eosin. For each section, the number of nuclei in the ONL was counted in the same region at 400 μm from each edge of the optic nerve over a 400 μm length (n=10 values for each eye). Since maximal survival was observed with the SODN, it was used in all further experiments.

### Rhodopsin Immunohistochemistry on Whole Flat-Mount Retinas

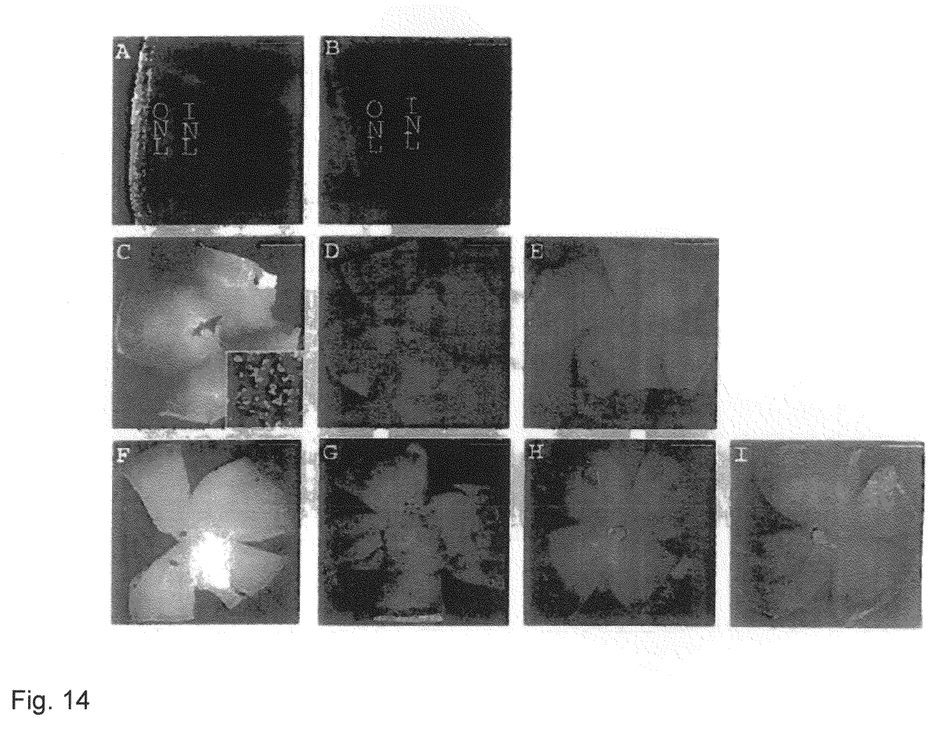

Rhodopsin immunohistochemistry performed at PN19 and 28 on flat-mount rd1/rd1 retinas of untreated (control) mice allows to follow the progressive loss of rhodopsin signal) see Results section (FIGS. 14C and 14E)). Therefore, rho-4D2 immunohistochemistry of PN28 whole flat-mounts was used as a global and rapid method to evaluate the potential rescue of photoreceptors and was therefore used to evaluate dosing effects and assayed of scrambled ODNs.

Rhodopsin immunohistochemistry was assessed on whole flat-mount retinas as previously described. Briefly, at PN19 and 28, ocular globes were fixed in 4% paraformaldehyde (Merck Eurolab) for 1 hour. Retinas were isolated, placed in PBS in 1.5 mL microcentrifuge tubes, permeabilized in PBS, 0.1% Triton X-100 (Sigma-Aldrich) for 5 min, and incubated in blocking buffer (PBS containing 0.1% bovine serum albumin (Sigma-Aldrich), 0.1% Tween 20 (Sigma-Aldrich) and 0.1% sodium azide (Sigma-Aldrich)) for 15 min. Retinas were incubated in rod photoreceptor specific monoclonal mouse antibody rho-4D2 (1/100 dilution in blocking buffer; kindly provided by Dr. Robert Molday, University of British Columbia, Vancouver BC, Canada. As negative controls, normal mouse serum (Nordic Immunological Laboratories, Tebu-bio, Le Perray en Yvelines, France) or mouse monoclonal antibody Leu-M5 directed against macrophages and monocytes (BD Biosciences, Pont-de-Claix, France) were used instead of rho-4D2 antibody (1/100 dilution in blocking buffer, 1 hour). Then, the retinas were washed 3 times for 5 min in blocking buffer and incubated with a secondary goal anti-IgG mouse antibody conjugated to Alexa Fluor 488 (1/250 dilution in blocking buffer; Molecular Probes, Leiden, Netherlands). The incubation volumes were 0.2 mL for antibody incubations and 1.5 ml for blocking and washing steps. After washing 3 times in PBS for 5 min, retinas were mounted in PBS-glycerol (1/1), with the photoreceptor layer facing up, and examined by fluorescence microscopy with a 2.5 objective and photographed using a digital SPOT camera (Optilas). All pictures were taken with an exposure time of 5 seconds. For each retina three pictures were taken to cover the whole retinal surface. Photographs of flat-mounts were merged in Photoshop 7.0 to reconstruct the whole retina.

Intensity of rhodopsin immunoreactivity was quantified by using the luminosity feature of Photoshop for raw pictures. Tissue and background regions were manually selected. Any residual pigmented epithelium was excluded. Mean pixel brightness was determined for each region by using the “Histogram” imaging feature. To normalize background levels among images, the mean brightness level per pixel of the tissue region was divided by the background region from each flat-mount image.

Follow-up experiments with the WTS ODN, the corresponding two scrambled ODNs (WTSscr7/scr25), and the untreated or PBS-treated controls were repeated three more times (6 eyes×3 for each condition). Other controls included injection of the WTS ODN without iontophoresis and iontophoresis without any injection (4 eyes for each condition).

The number of treatments was assayed by injection of the WTS ODN (1 μL of 500 μM) following iontophoresis performed once at PN4, twice at PN4 and 6, three times at PN4, 6, and 8 or three times at PN6, 8 and 10 (4 eyes for each condition).

Further Evaluation of Phenotypic Changes Observed in the Retina of rd1 Mice Treated with S-ODN

### Rhodopsin Immunohistochemistry on Eye Sections

Eyes of PN28 wild-type (4 eyes), PBS (6 eyes), or ODN-treated (8 eyes) rd1/rd1 mice were enucleated, quickly frozen in Tissue-Tek OCT-compound (Bayer Diagnostics), and sectioned (10 μm). For each eye, sections that included optic nerve were stained with hematoxylin and eosin for structural analysis. Sections were fixed in 4% paraformaldehyde (Merck Eurolab) for 5 min at room temperature, washed in PBS, and incubated 1 hour in mouse rho-4D2 antibody (1/100 dilution in PBS). As negative controls, normal mouse serum (Nordic Immunological Laboratories) or mouse monoclonal antibody Leu-M5 (BD Biosciences) replaced the primary antibodies (1/100 dilution in PBS). Slides were washed 3 times in PBS and incubated with mouse anti-IgG conjugated b Alexa Fluor 488 (1/250 dilution in PBS; Molecular Probes). Then, the slides were washed 3 times in PBS, mounted in PBS/glycerol (1/1) and examined under a fluorescence microscope (Leica).

### β-PDE Immunohistochemisty on Eye Sections

The presence of β-PDE was assessed in eye sections from PN28 untreated and PBS- or ODN-treated rd1/rd1 mice previously labeled with rho-4D2. Immunohistochemistry was performed with rabbit IgG PDE6β antibody (1/100 dilution; Affinity Bioreagents, Golden, Colo., USA) or non-immune rabbit serum (1/100 dilution) as primary antibodies and goat anti-IgG rabbit antibody conjugated to Texas red Fluor (1/100 dilution; Molecular Probes) as secondary antibody. Sections were mounted in PBS/glycerol (1/1) and examined under a fluorescence microscope (Leica).

Genotypic Changes Induced in rd1 Retina Treated with the S-ODN

Genomic DNA from rd1/rd1 retinas was extracted from individual whole flat-mount retinas (PN28) using a DNeasy Tissue kit and eluted in 200 μL of AE buffer as per manufacturer instructions (Qiagen, Courtaboeuf, France and Valencia, Calif., USA). Genomic DNA from wild-type retinas, untreated, and PBS-treated rd1/rd1 retinas served as controls.

Allele-specific real-time PCR was used to detect small amounts of wild-type sequence resulting from ODN treatment. DNA samples isolated from treated and untreated retinas were used as template DNA in PCR reactions with primers designed to preferentially amplify wild-type rather than mutant β-PDE sequence. The 3′ base of one primer was complementary to wild-type sequence, but not rd1/rd1 sequence, at position 1048 of GenBank accession no. X60133. Primers used for preferential amplification of wild-type β-PDE sequence were 5′-TGCAAGCATTCATTCCTTCGAC-3′ (SEQ ID NO:10) and 5′-AAGCCACTTTCTGCTACG-3′ (SEQ ID NO:11). For normalization calculations, parallel reactions using aliquots of the same source of template DNA were run using primers designed to nonspecifically amplify β-PDE sequence:

| | |
|---|---|
| 5′-CATCCCACCTGAGCTCACAGAAAG-3′ | (SEQ ID NO:12) |
| and | |
| 5′-GCCTACAACAGAGGAGCTTCTAGC-3′ | |

Reactions were run in a Bio-Rad iCycler iQ real time PCR detection system with melt curve analysis (Bio-Rad, Hercules, Calif.). Reactions of 20 μl final volume included template DNA (10 ng), primers (50 nM), and QuantiTect SYBR Green PCR Master Mix (Qiagen, Valencia, Calif.), which is composed of SYBR Green I (a dye that fluoresces strongly when bound to dsDNA), HotStarTaq DNA polymerase, dNTPs, and buffer components optimized by the manufacturer. Poly (dI:dC), 20 ng per assay, was added to reduce nonspecific PCR products.

The limit of detection for quantification was considered 10 times the root mean square noise of fluorescence intensity across a window usually spanning cycles 2 through 10. The cycle number at which product accumulated past this detection threshold (Ct) was related to beginning copy number of a specific template allele in a reaction by a calibration curve created with standard amounts of the wild-type β-PDE gene. A lower Ct compared to untreated rd1/rd1 controls indicates the presence of a specific allele, in this case, a presumed rd1/rd1 allele repaired to wild-type sequence (mutant adenine converted to wild-type cytosine). For each template DNA, the Ct from either mutant or wild-type allele specific β-PDE reactions were subtracted from Ct of a non allele specific reaction to correct for differences in total DNA starting concentrations. Normalizing reactions were identical to β-PDE reactions with the exception that PCR primers specific to the 18s-RNA gene were used to amplify template DNA. Ct data are means±SEM of 5-6 experimental samples assayed in 5-10 replicates. To determine the efficiency of the assay, calibration curves of gene copy versus threshold cycle were made using increasing amounts of wild-type genomic DNA (1 μg to 10 ng) mixed with 10 ng of rd 1/rd1 genomic DNA.

In separate control assays, various repair ODNs (containing wild-type sequence) were added into DNA template samples at several concentrations to determine whether their presence caused artificial decreases in Cts. Their presence had no effect on Cts (data not shown) over a wide range of concentrations.

### Statistical Analysis

Results were expressed as means±SD and compared using the non-parametric Mann Whitney test and the analysis of variance (ANOVA) test with post-hoc Student-Newman-Keuls. P<0.05 was considered as significant.

### Results

Enhanced Delivery of Labeled-Oligonucleotides to Retinal Cells of rd1/rd1 Mouse Eyes Using Iontophoresis Prior to Intravitreal Injection

Transpalpebral iontophoresis was performed with an eye-glass-shaped electrode made with aluminum foil and sponge (FIG. 12A) and connected to a power supply (FIG. 12B). It did not cause any detectable clinical or histologic lesions to the mice eyes (FIG. 12C). One hour after the intravitreal injection of CY3-labeled WTS ODN, without any current applied, fluorescence was observed in the ganglion cell layer (GCL) and in a few cells within the inner nuclear layer (INL). No fluorescence was detected in the outer nuclear layer (ONL) of PN7 mice (FIG. 12D). In contrast, when saline iontophoresis was applied immediately prior to the ODN injection, intense fluorescence was observed both in nuclei of the INL and the ONL (FIG. 12E). No fluorescence was observed in any retinal layers of PBS-treated eyes (FIG. 12F) or non-injected control eyes (with or without iontophoresis) (data not shown) (8 eyes for each condition).

Detection of β-phosphodiesterase Protein on Eye Sections at PN28

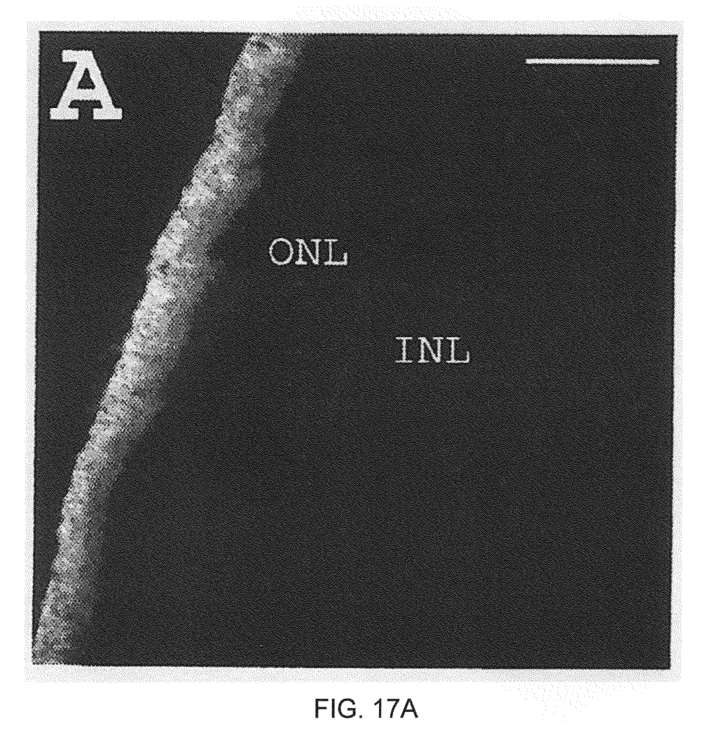

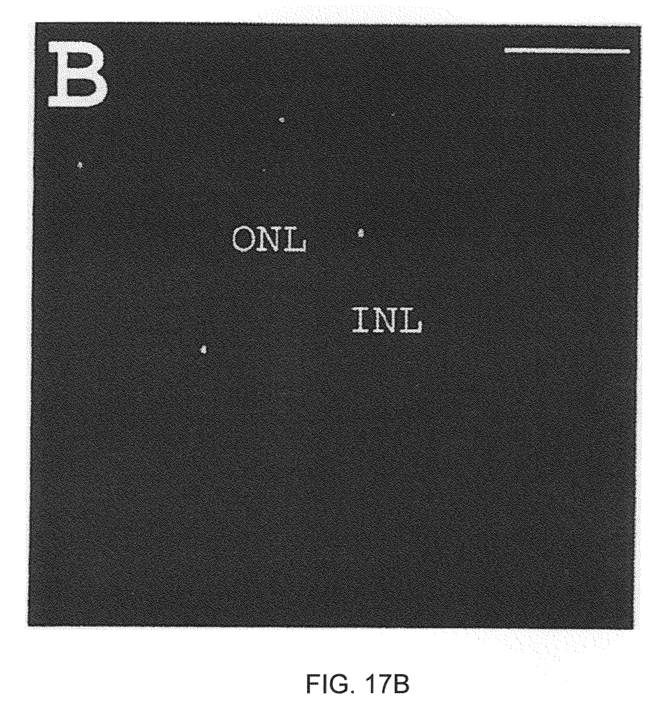

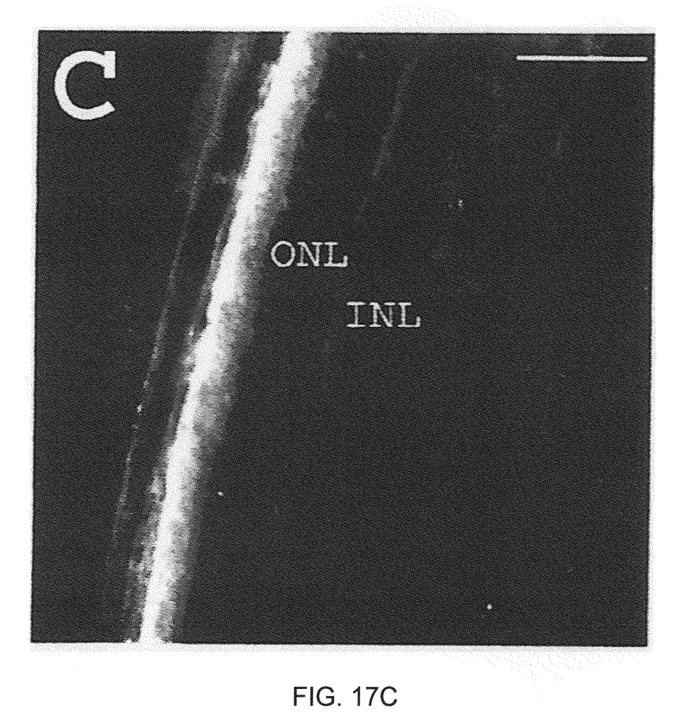

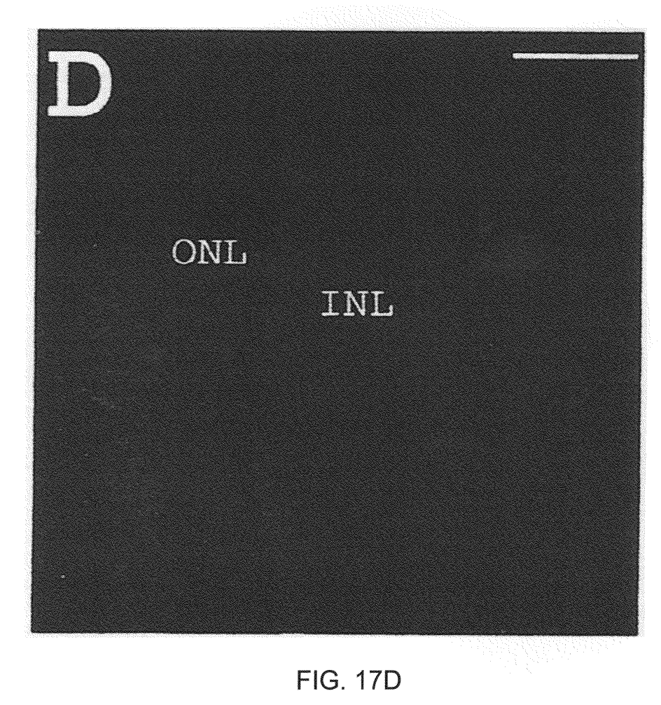

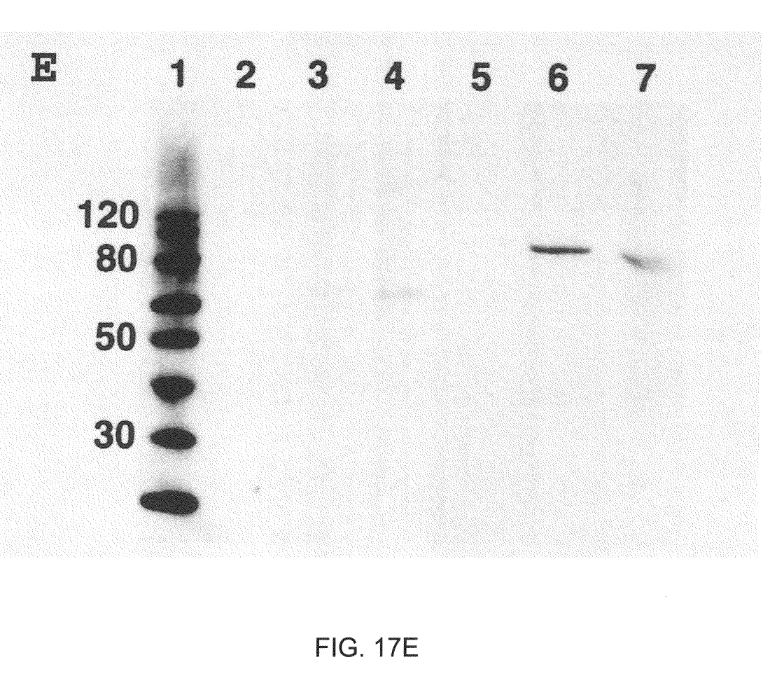

The antibody directed against β-PDE specifically labeled the rod outer segments in the wild-type mice at PN28 (FIG. 17, top panels). The specificity of the anti-β-PDE antibody was confirmed by Western blot with a specific signal on wild-type retinas and an absence of signal on rd1/rd1 retinas (FIG. 17E).

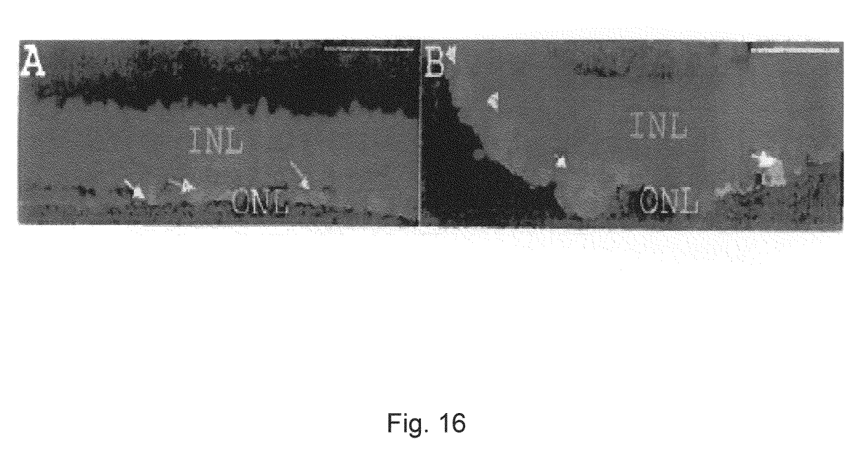

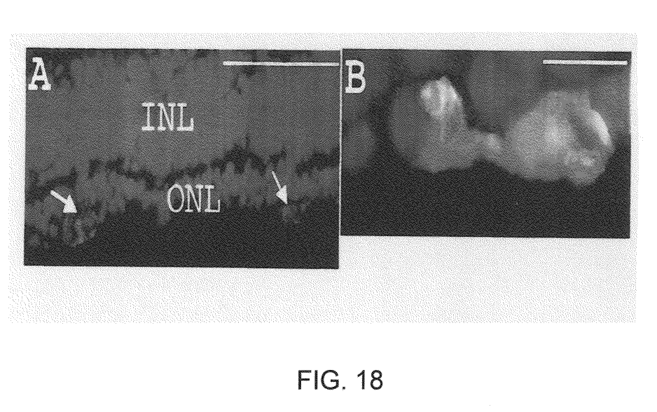

In the rd1/rd1 mouse, β-PDE immunoreactivity was not present at any stage. However, in the 500 μM WTS ODN-treated rd1/rd1 eye sections, a positive fluorescent signal for β-PDE was observed (FIG. 18A). An average of 26±6 β-PDE immunopositive cells were detected on whole sections from ODN-treated flat-mount retina (3.5 mm long by 10 μm thick). The 3.5 mm by 10 μm section corresponds to a surface area of 0.035 mm2, or 743 positive cells per mm2. The estimated surface area of a retina is two-thirds the surface of a sphere (4πr2), and for a radius of 1.25 mm at PN28, this is 13.1 mm2. The entire retina therefore has roughly 743×13.1=9730 β-PDE positive cells. Double labeling with rho-4D2 showed that cells positive for β-PDE were also positive for rhodopsin (FIG. 18B). However, rhodopsin expressing cells were more numerous than those expressing β-PDE (FIGS. 16B and 18A). No significant fluorescence was observed when ODN was omitted in otherwise complete treatments (data not shown).

Analysis of Conversion of Genomic DNA from rd1/rd1 to Wild-Type

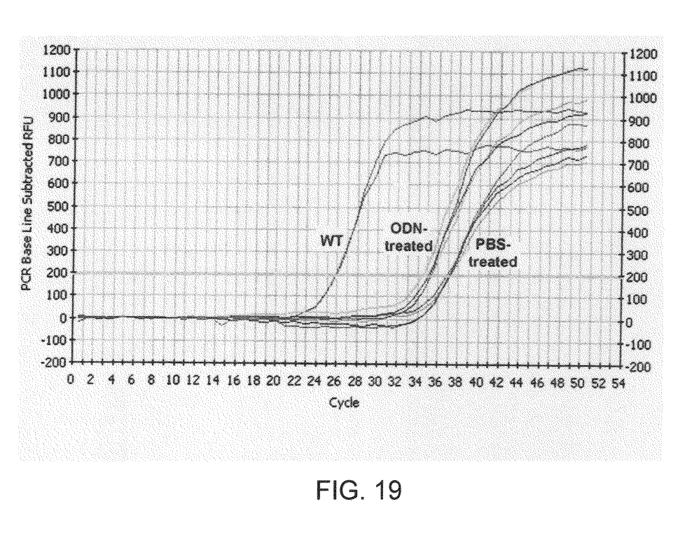

DNA extracted from ODN- or PBS treated rd1/rd1 retinas and from wild-type retinas was used as template in allele-specific real-time PCR with primers designed to amplify only wild-type DNA. The Threshold Cycle values (Ct) were 35±0.6 (6 retinas) for the ODN-treated group and 37±0.5 (5 retinas) for the negative control PBS group. The Ct for the same amount of pure wild-type genomic DNA was 26±0.1 (6 retinas) (FIG. 19). Treatment of the rd1/rd1 mice, as performed in our study, leads to a significantly lower Ct compared to the negative control PBS group (unpaired t-test; p=0.0334), indicating that the ODN-treated group contained wild-type DNA and suggesting that treatment induced repair of genomic DNA in rd 1/rd1 mice. Based on the efficiency analysis, the effect of treatment on the appearance of wild-type β-PDE DNA copy number was taken as 1.95(a-b) where a=Ct for ODN treatment and b=Ct for wild-type genomic DNA. The 1.95(a-b) value divided into 100% provides the percent conversion of the mutant adenine to wild-type, i.e., 100/1.95(a-b)=100/1.95(35−26)=0.2%.

### Discussion

The results provide evidence that gene repair in photoreceptor cells is feasible when iontophoresis is combined with intravitreal injection of a phosphorothioate single-stranded ODN. β-PDE gene repair was detected in 0.2% genomic DNA of treated rd1/rd1 retinas. Phenotypically, this repair was ascertained by the expression of β-PDE and rhodopsin proteins in treated rd1/rd1 mice retinas at PN28, a stage when untreated or mock treated mice do not express any β-PDE or significant rhodopsin. Despite the relatively low number of converted copies of genomic DNA, a significant rescue of photoreceptors was observed. The present work is a proof of concept exercise that demonstrates the feasibility of this non-viral, ODN-targeted gene repair strategy in the neural retina.

For targeted gene repair, the adequate delivery of a sufficient amount of ODNs to the target cells is critical. In the eye, the direct intravitreal injection does not allow enough intact ODNs to reach photoreceptor nuclei. As shown by the distribution study, the application of iontophoresis prior to the injection of ODNs results in an increased penetration of ODNs in photoreceptor nuclei. When performed after iontophoresis, the repetition of injections two or three times induces a significant rescue of photoreceptors. Therefore, photoreceptor cell rescue requires repeated delivery and/or a critical mass of intravitreal ODNs combined with an enhanced penetration efficiency to the target cells. These observations may explain the failure of previous attempts to achieve a repair of the rd1/rd1 gene by simple injections (unpublished observations of J H Boatright and J M Nickerson). Others studies have shown that iontophoresis increases intracellular penetration of small gene fragments. However, it may be surprising that, in the present study, iontophoresis before intravitreal injection worked as well as iontophoresis after injection. An explanation might be that iontophoresis in our experiment is not critical for moving DNA, but instead iontophoresis may boost ODN transport by electroosmosis or by current-induced changes within the cell membrane organization and charges. In the skin, transport during post iontophoretic periods has been described, and the use of saline iontophoresis prior to drug application was previously shown to increase drug penetration. Further experiments will be needed to understand this phenomenon.

Increased Number of Photoreceptors Observed on Retina Sections at PN28 from rd1/rd1 Treated Mice with Specific ODNs

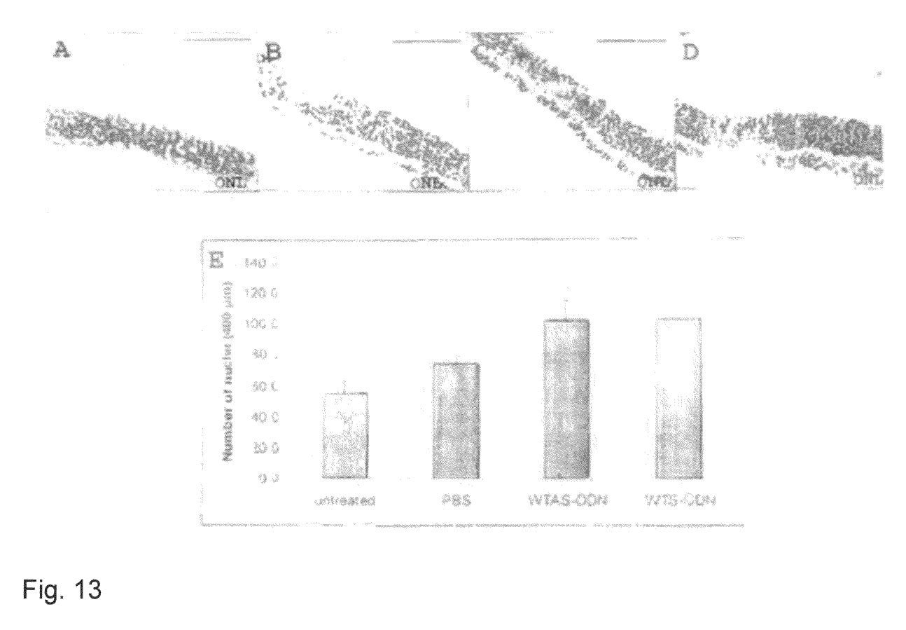

While a single row or less of sparse cells was observed at PN28 in the ONL of untreated rd1/rd1 retina (FIG. 13A), discontinuous areas containing two or three rows of cells were observed over the entire ONL of mice treated with WTAS ODN and WTS ODN, FIGS. 13C and 13D represent two of those areas where maximal rescue was observed. In PBS-treated eyes, a limited increase of cells was observed in the ONL (FIG. 13B). At PN28, the number of cells in the ONL was not significantly different in WTAS ODN- and WTS ODN-treated retinas with previously described conditions, (respectively 101±15 and 103±14 on a 400 μm length; 8 eyes for each condition, P>0.05). But there were significantly increased when compared to the PBS-treated retinas (74±5; 6 eyes) or to the untreated retinas from rd1/rd1 mice (55±8; 6 eyes) (P<0.05 and P<0.01 respectively).

### Rhodopsin Immunostaining at PN28 was Increased by Specific Oligonucleotides

Extensive positive immunoreactive signal for rhodopsin was observed in wild-type eye sections at PN28 when rho-4D2 is used as primary antibody (FIG. 14A). Sections reacted with normal mouse serum in place of rho-4D2 yielded negative results (FIG. 14D). In rd1/rd1 flat-mount retinas, rhodopsin-positive signal was observed throughout at PN19 (FIG. 14C). The intense fluorescence observed at low magnification at PN19 corresponds to dispersed immunoreactive photoreceptors as shown at a higher magnification (inset in FIG. 14C). No positive signal was detected when normal mouse serum was used as control on rd1/rd1 lat-mount retinas at PN19 (FIG. 14D). At PN28, the rhodopsin signal was extremely low, reflecting the advanced and nearly complete degeneration of rods in these retinas all this time point (FIG. 14E), paralleling the time course of the rd1/rd1 retinal degeneration.

Rhodopsin is the most abundantly-expressed photoreceptor-specific protein and is frequently used as a marker to detect the existence of rod photoreceptor cells. To evaluate the potential of sense phosphorothioate ODNs to induce gene correction and subsequent photoreceptor survival, rhodopsin immunohistochemistry was performed on treated flat-mount rd1/rd1 retinas at PN28. Gene repair treatment, consisting of iontophoresis followed by injection with 1 μL of 500 μM specific ODNs, was performed on rd1/rd1 mice at PN4, 6, and 8. As shown in Table 1, the calculated ratios of tissue-to-background fluorescence (Tissue F1/Bkgd F1) were less than 1.75 for no treatment, treatment with PBS alone, or treatment with any of the scrambled ODNs (WTSscr25, and WTSscr7; ODN nomenclature is given in the legend of Table 1). Treatment with WTS ODN yielded the most intense rhodopsin immunostaining, resulting in a reproducible Tissue F1/Bkgd F1 ratio of 2.57 (FIG. 14F). This increase was statistically significant compared to all other treatments as determined by the non parametric Mann-Whitney test (P<0.004 for comparisons between WTS and any other treatment groups).

When the WTS ODN injection was not coupled to the application of current (FIG. 14G) or when the current was applied without any intravitreal injection (FIG. 14H), no effect on rhodopsin immunostaining was observed. Moreover, phosphorothioate sense scrambled ODNs (WTSscr25 and WTSscr7) did not induce any increase in rhodopsin immunostaining (Table 1, FIG. 3I, P>0.05, compared to PBS treatment), demonstrating the specificity of the response to the combined iontophoresis/injection treatment with WTS ODN.

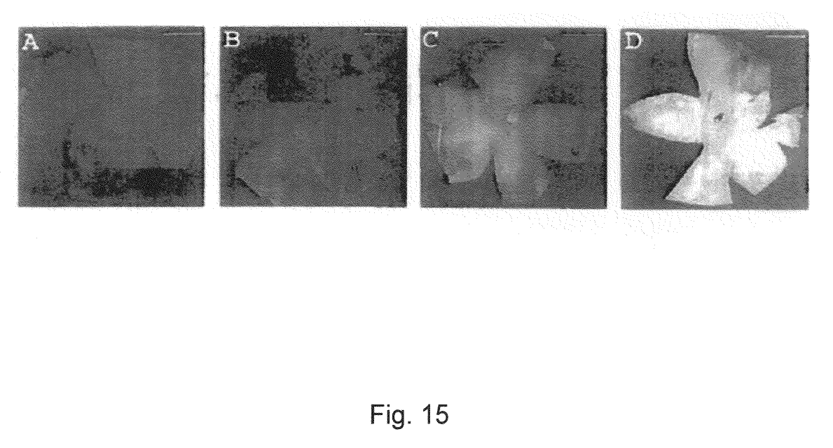

While one single combined treatment at PN4 had no effect on photoreceptor survival, two combined treatments at PN4 and 6 induced a detectable increase of rhodopsin immunostaining (Table 2, FIG. 15). Photoreceptor survival, as evaluated by rhodopsin immunostaining on whole flat-mount retinas, was significantly increased by three successive treatments with 1 μL of the 500 μM WTS ODN. Performing treatments at PN6, 8, and 10 provided a rescue similar to that observed when treatments were delivered at PN4, 6, and 8.

The application of three successive treatments (iontophoresis prior to injection) at PN4, 6, and 8 with 1 μL of 500 μM WTS ODN had a significant effect on rhodopsin expression in PN28 rd1/rd1 mice, indicating increased photoreceptor survival. These conditions were used for all further experiments.

### Rhodopsin Immunostaining on Eye Sections at PN28

Clusters of rhodopsin positive cells in multiple rows were homogenously dispersed across the retinas of mice treated with WTS ODN (FIG. 16B), while some cells in a single row of the residual ONL were labeled in PBS-treated eyes (FIG. 16A), demonstrating that treatment by iontophoresis followed by injection of specific ODNs induced the survival of rod photoreceptors.

The type of ODN is one of the other critical issues in gene repair. In the model, the sense ODN (targeting the transcribed strand of genomic DNA) and antisense ODN (targeting the non-transcribed strand of genomic DNA) induced a roughly equivalent genomic repair. Several studies have shown that the polarity of ODNs influences their targeted repair efficacy. In the earlier in vitro studies, the antisense ODN was found more effective than the sense ODN for inducing gene repair. However, the superior efficacy of the antisense ODN is not universally observed; recent in vitro and in vivo studies have found the sense ODN to be significantly more effective. Several factors such as the transcription activation, the phase of the cell cycle and/or genomic sequences surrounding the target mutation may influence the strand bias. Herein is developed an in vitro model allowing the study of the importance of the target DNA sequence in gene repair. It is based on the introduction of different target sequence at a single identical genomic site in 293 cells. Using this model, it is shown that strand bias is sequence specific. When targeting by gene repair the sequence containing the rd1 mutation in this model, antisense and sense ODNs were also roughly equivalent. Although this in vitro model was not realized on retinal cells, it will be useful to analyze the factors potentially influencing the frequency of rd1 correction. The selection of optimized parameters will be applied in vivo to enhance the repair of the rd1 mutation.

The minor effect of PBS or scrambled ODNs treatment on photoreceptor survival as observed on flat-mount retinas rhodopsin immunohistochemistry compared to untreated control (sec Table I) may be attributed to the induction of endogenous neurotrophic factors, known to delay the retinal degeneration in the rd1/rd1 mouse model. Such an effect has been detected in eyes following surgical interventions and other forms of mock, sham, or vehicle control treatments. Possibly a neurotrophic effect may also explain the much larger number of rescued rhodopsin positive cells than β-PDE positive cells. Thus, a strategy combining our therapeutic approach with the additional use of neurotrophic factors may potentiate the effect of low genomic repair.

These results have to be taken as a proof of concept and cannot be extrapolated to the human retinal dystrophy. In the rd1/rd1 mice, retinal degeneration begins soon after birth and progresses quickly allowing for a narrow window of opportunity for effective gene repair. Furthermore, in this mouse model, an extensive and very rapid degenerative process results in a toxic environment for corrected cells. In human, most of the degenerative diseases progress slowly, allowing for a longer period of therapeutic window. At the present, other models of slowly progressing retinal degeneration are being evaluated in our laboratory. Using these models, it is possible to determine whether applying a larger number of treatments will increase targeted gene repair efficiency and whether gene repair allows for a permanent rescue of photoreceptors.

In conclusion, this study provides a proof of principle for non-viral targeted gene repair in photoreceptors of the rd1/rd1 mouse using combination of iontophoresis and intraocular injection of specific ODNs and opens new avenues for the treatment of ocular degenerative and blinding eye diseases.

### Abbreviations

β-PDE: β-subunit of rod photoreceptor cGMP-phosphodiesterase, PN: postnatal day, ODN: oligonucleotide, GCL: ganglion cell layer, INL: inner nuclear layer, ONL: outer nuclear layer, CY3: cyanin 3, PBS: phosphate buffer saline, chim: chimeric, WTS/AS: wild-type sense/antisense, o: 2′-O-methyl RNA bases, 25 or 45: 25 or 45 mers, PO: phosphodiester, polyU: polyU-protected, 6x6PS: six phosphorothioate linkages at each 5′ and 3′ positions, scr7/25: scrambled sequence of the 7 central bases/the entire 25 mers ODN.

### Example 4

### Transpalpebral Iontophoresis Combined with Oligonucleotides Intravitreous Injection Allows the Targeting of Photoreceptor Cells In Vivo

Inherited retinal degenerations result in progressive photoreceptor loss and can be caused by punctual nonsense mutation in β-PDE gene. Targeting gene repair is a new gene therapeutic that can corrects a punctual mutation by targeting oligonucleotide (ODNs) to the genomic DNA sequence where alteration is required. One of the main limiting step in this technique is the need for an efficient delivery of high ODN amounts in the targeted cells. After intravitrous injection, ODNs concentrate in inner nuclear layers and finally accumulate in RPE cells. However, their penetration in the photoreceptor nuclei is very poor, compromising any potential gene repair.

Since iontophoresis increases the intracellular penetration of ODN in the cornea, the present study evaluated the possibility to associate intravitreous injection of ODNs with the application of iontophoretic current to optimize the intra-retinal delivery of ODNs in the pup's mice retina.

### Materials and Methods

The materials and methods of this study are essentially as described in Examples 2 and 3 above. Saline transpalpebral iontophoresis associated with intravitreous injection of red-labeled ODNs encoding for the sense wild-type β-PDE gene sequence were applied to rd1/rd1 mice homozygous for the nonsense mutation (amino acid position 347) in the β-PDE gene at post natal day 7.1 μL of ODN (500 μM) was injected into the vitreous with a micro injector and controlled with a microscope.

Various treatment parameters were tested. Cationic or anionic transpalpebral iontophoresis (respectively positive or negative electrode connected to the eyelids) was performed immediately before or after the intravitreous injection of ODN labeled using a current of 1.5 mA for 5 min (4 eyes for each combination). Conditions with lower current intensities (0.5 mA for 5 min) were also tested (4 eyes for each condition). The effect of timing (1, 3 and 6 hours) between iontophoresis and ODN intravitreous injection was carried out (4 eyes for each condition). For the control groups, rd1/rd1 mice at PN7 received either iontophoresis followed by PBS injection, ODN injection without iontophoresis or had no treatment (4 eyes for each condition). Fluorescence intensity of the outer nuclear layer fluorescence was quantified by image analysis of cryosections. Three microphotographs taken from 5 sections from each eye with a fixed exposure time and expressed in mean pixel brightness ±SD served as the basis for this analysis. Relative OLN intensity values obtained were compared and analyzed by statistic program.

### Results

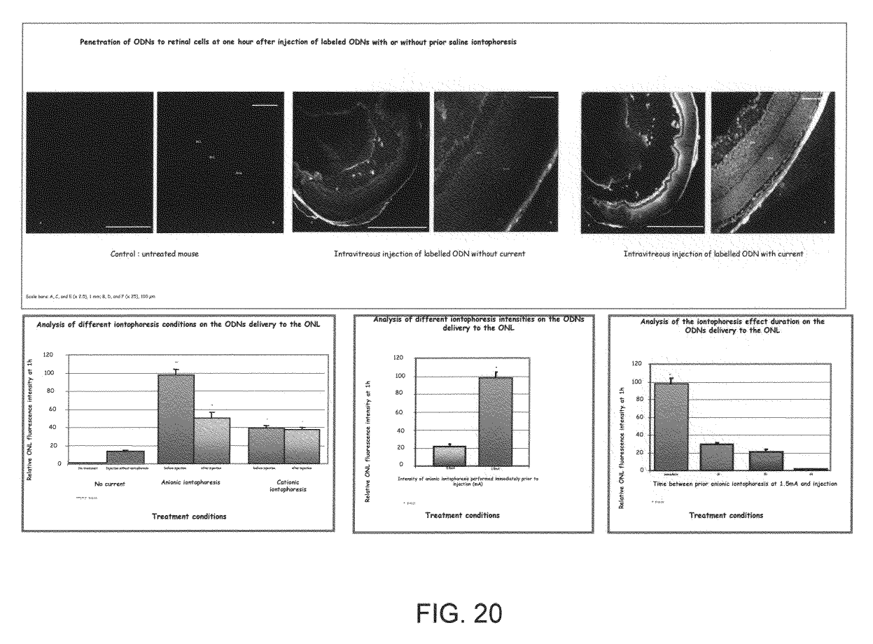

All conditions of transpalpebral saline iontophoresis increase the penetration of ODNs in the retinal layers when compared to injection without iontophoresis. Intact ODNs were extracted from the retina at one hour after treatment. As illustrated in FIG. 20, photoreceptors targeting was significantly increased when the saline transpalpebral iontophoresis is applied before ODN injection as compared with its application after ODN injection. Anionic iontophoresis is more efficient than cationic iontophoresis. Of all tested conditions, optimal photoreceptor targeting is achieved with application of transpalpebral saline anionic iontophoresis using 1.5 mA for 5 min before ODN injection. The facilitation of intraocular penetration by iontophoresis remains apparent at least for 3 hours with a marked decrease observed later. No intraocular tissue damage was observed after the iontophoresis application.

### Example 5

### Genoplasty in rd1 Mice In Vivo

Mice homozygous for the rd1 mutation display hereditary retinal degeneration and serve as a model for human retinitis pigmentosa. rd1 mice present a C→A mutation in the rod c-GMP-phosphodiesterase (PDE) β-subunit gene, which creates a stop codon leading to the truncation of the protein. In these animals, the retinal rod photoreceptor cell death begins at about postnatal day 8 and complete degeneration is achieved by 4 weeks.

The purpose of this work is to evaluate the genoplasty strategy on the rd1 mice and study the intra-retinal penetration and action of different types of ODNs designed to correct the rd1 mutation.

### Materials and Methods

The materials and methods of this study are essentially as described in Examples 2 and 3 above.

### Results

### Enhanced Delivery of Injected Labeled-Oligonucleotides to Retinal Layers by Iontophoresis: a Process Delivering Drugs Using Low Current Density, in a Safe Way

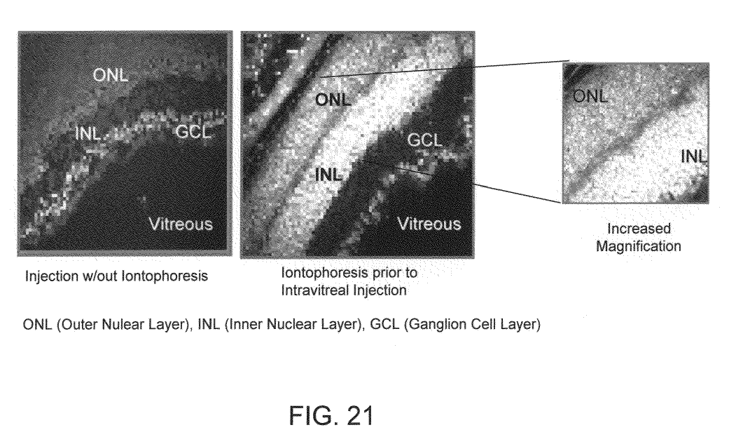

Delivery of injected labeled-oligonucleotides to retinal layers by iontophoresis was detected by fluorescent microscopy of CY3-labeled oligonucleotides (approx. 8 kDa) in eye sections one hour after intravitreal injection following or not iontophoresis. As illustrated in FIG. 21, iontophoresis was very efficient to target retinal cells.

### Comparative Capacity of Specific Chimeric, Phosphodiester, Phosphorothioate and Poly U-Protected Oligonucleotides Targeting Coding or Non-Coding DNA Strand to Correct the rd1 Mutation in Mice

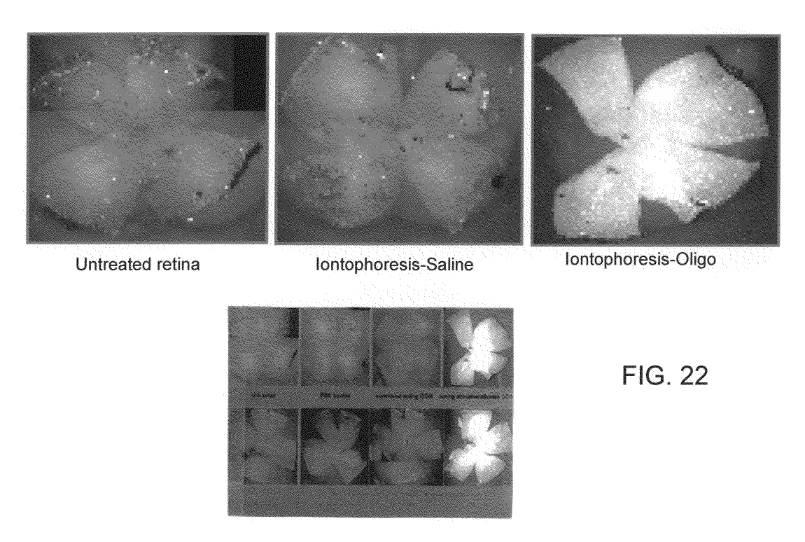

Specific chimeric, phosphodiester, phosphorothioate and poly U-protected oligonucleotides were injected in rd1 mice following iontophoresis at day post natal 4, 6 and 8. Photoreceptor survival evaluated by opsin-immunohistochemistry on whole flat-mounted retinas indirectly reflecting potential genomic correction of rd1 mutation. Retinas from treated mice at PN28 were used for opsin-immunohistochemistry using Rho-4D2. As illustrated in FIG. 22, the best results were obtained with specific coding phosphorothioate oligonucleotide showing a dose-dependant rescue of photoreceptors.

### Detection of β-Phosphodiesterase Protein on Eye Sections of Treated rd1 Mice

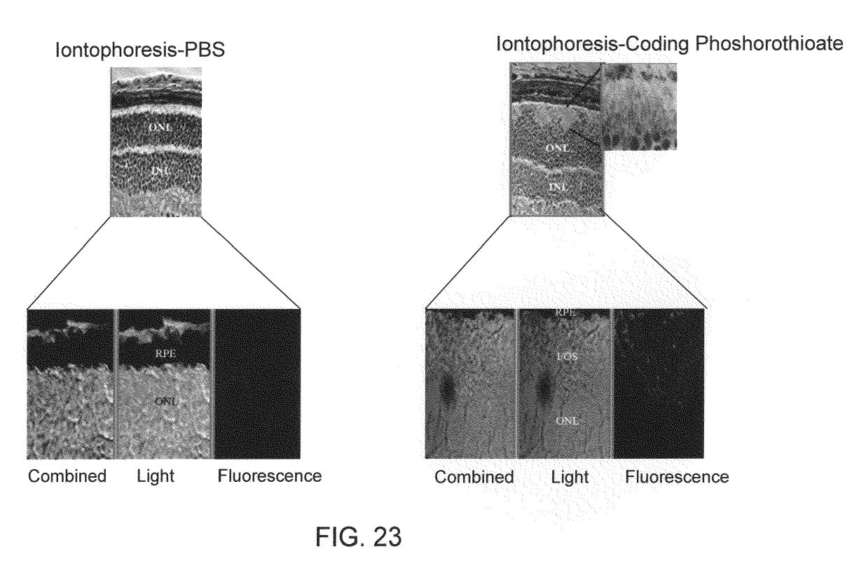

Treated rd1 mice were injected by coding phosphorothioate oligonucleotides following iontophoresis at PN 4, 6 and 8. Eye sections from mice at PN12 were used for immunohistochemistry anti-β PDE (Affinity Bioreagents) using a secondary antibody conjugated to Alexa Fluor 488. They were examined under a Confocal microscope. As illustrated in FIG. 23, β-phosphodiesterase protein was detected in eye sections from mice after treatment. The enhanced retinal expression of β-phosphodiesterase protein in treated rd1 mice further supports iontophoresis mediated retinal delivery.

A qualitative evaluation of iontophoresis of labeled oligonucleotide on rat is also provided in FIG. 24.

### Analysis of Correction Rate for rd1 Mutation on Genomic Mice DNA

DNA was extracted at PN28 from ODN- or PBS-treated mice retinas. DNA was used as template in real-time PCR with allele-specific primers designed to amplify only wild-type β-PDE DNA. Control PCRs were run with non allele-specific β-PDE primers to compensate for any errors in template concentration measurement.

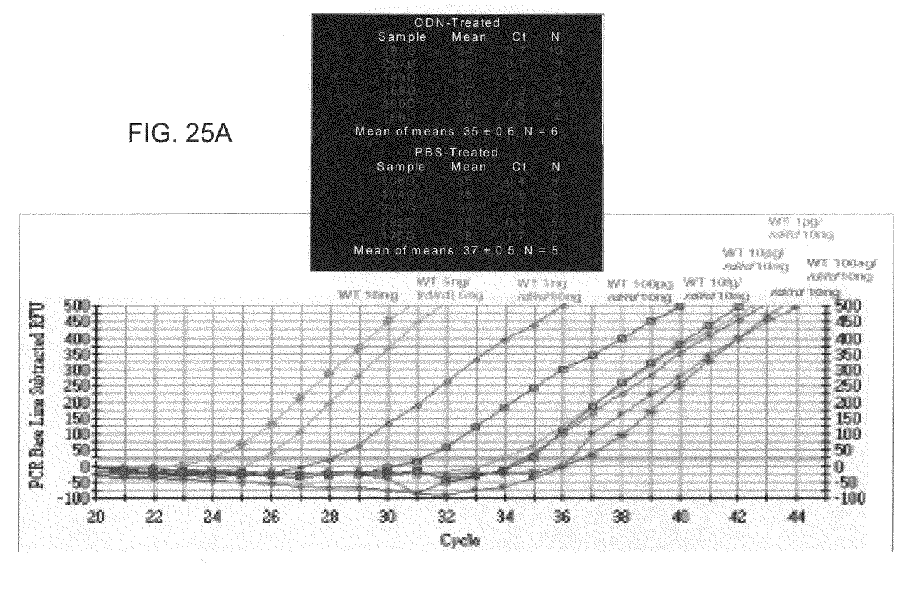

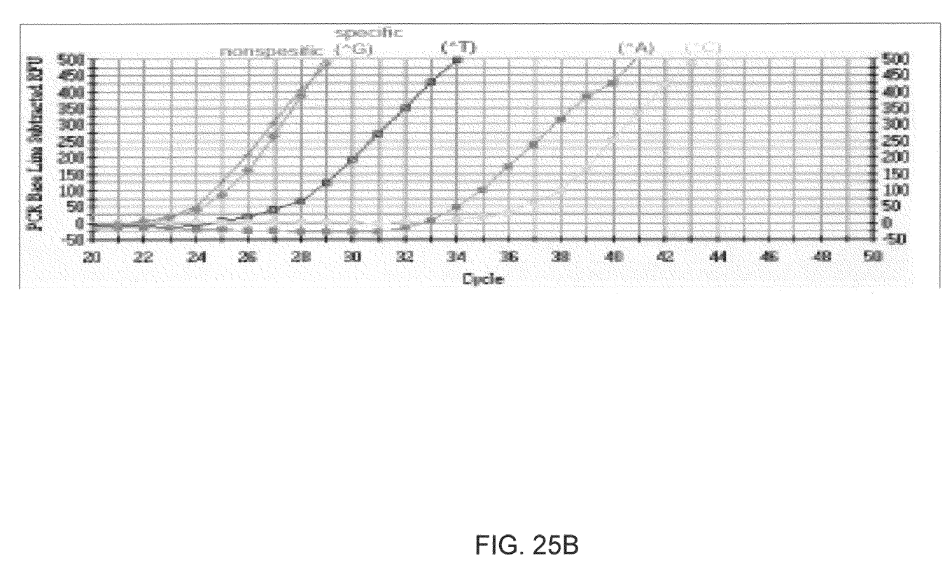

As predicted and as illustrated in FIG. 25, reactions with wild-type β-PDE DNA reached threshold in many fewer cycles than those with untreated rd template. A SRT-PCR analysis shows statistically significant leftward shift in the reaction profiles of ODN-treated samples, indicating that treatment induces repair of genomic DNA.

### ADDITIONAL REFERENCES

Agarwal, S. et al., Biomol. Eng., 20:7-20, 2003
Alexeev, V. et al., Nat. Biotechnol., 8:43-7, 2000
Alexeev, V. & Yoon, K., Pigment Cell Res., 13:72-9, 2000
Alexeev, V. et al., Gene Ther., 9:1667-75, 2002
Andrieu-Soler, C. et al., Nucleic Acids Res., 33:3733-42, 2005
Asahara, T. et al., Jpn. J. Opthalmol., 45:31-9, 2001
Auricchio, A., Vision Res., 43:913-8, 2003
Baehr, W. et al., FEBS Lett., 278:107-14, 1991
Bandyopadhyay, P. et al., J. Biol. Chem., 274:101 63-72, 1999
Barilett, R. et al., Nui. Biotechnol., 18:6 15-22, 2000
Barza, M. et al., Invest. Opthalmol. Vis. Sci., 28:1033-6, 1987
Behar-Cohen, F. et al., Exp. Eye Res., 65:533-45, 1997
Behar-Cohen, F. et al., Invest. Opthalmol. Vis. Sci., 39:897-904, 1998
Behar-Cohen, F. et al., J. Fr. Ophtalmol., 24:319-27, 2001
Behar-Cohen, F. et al., Exp. Eye Res., 74:51-9, 2002
Berdugo, M. et al., Antisense Nucleic Acid Drug Dev., 13:107-14, 2003
Bertoni, C. & Rando, T., Hum. Gene Ther., 3:707-18, 2002
Blanks, J. et al., J. Comp. Neurol., 156:95-106, 1974
Brachman, E. & Kmiec, E., Genetics, 163:527, 2003
Caffe, A. et al., Invest. Opthalmol. Vis. Sci., 42:275-82, 2001
Cao, W. et al., Exp. Eye Res., 65:241-8, 1997
Cayouette, M. et al., Neurobiol. Dis., 6:523-32, 1996
Cayouette, M. & Gravel, C., Hum. Gene Ther., 8:423-30, 1997
Cole-Strauss, A. et al., Science, 273:1386-9, 1996
Curdy, C. et al., Eur. J. Pharm. Biopharm., 53:15-21, 2002
Davies, J. et al., Mol. Vis., 9:569-78, 2003
Drummond, P., Br. J. Clin. Pharmacol., 54:45-50, 2002
Faber, D. & Danciger, M., Curr. Opin. Neurobiol., 7:666-73, 1997
Frasson, M. et al., Invest. Opthalmol. Vis. Sci., 40:2724-34, 1999
Frasson, M. et al., Nat. Med., 5:1183-7, 1999
Guy, R. et al., J. Control Release, 64:129-32, 2000
Halhal, M. et al., Exp. Eye Res., 78:751-7, 2004
Hughes, L & Maurice, D., Arch. Opthalmol., 102:1825-9, 1984
Igoucheva, O. et al., Gene Ther., 8:391-9, 2001
Kalia, Y. et al., Adv. Drug Deliv. Rev., 56:619-58, 2004
Kim, A. et al., Pharm. Res., 10:1315-20, 1993
Kren, B. et al., Nat. Med., 4:285-90, 1998
Kren, B. et al., Proc. Natl. Acad. Sci. USA, 96:10349-54, 1999
Kren, B. et al., Semin. Liver Dis., 19:93-104, 1999
Kren, B. et al., Gene Ther., 9:686-90, 2002
Lam, T. et al., Arch. Opthalmol., 107:1368-71, 1989
Lachaud, J., Bull. Soc. Ophtalmol. Fr., 65:84-9, 1965
Laird, D. & Molday, R., Invest. Opthalmol. Vis. Sci., 29:4 19-28, 1988
LaVail, M. & Sidman, R., Arch. Opthalmol., 91:394-400, 1974
LaVail, M. et al., Invest. Opthalmol. Vis. Sci., 39:592-602, 1998
Li, S. et al., Invest Opthalmol. Vis. Sci., 45:1224-31, 2004
Li S. et al., Int. J. Pharm., 273:9-22, 2004
Liu, L. et al., Nucleic Acids Res., 29:4238-50, 2001
Liu, L. et al., Mol. Clin. Biol., 22:3852-63, 2002
Lu, I. et al., Gene Ther., 10: 1910-6, 2003
Marro, D. et al., Pharm. Res., 18:1701-8, 2001
Naik, A. et al., Pharm. Sci. Technolo. Today, 3:318-326, 2000
Nakamura, M. et al., Gene Ther., 11:838-46, 2004
Nugroho, A. et al., Pharm. Res., 21:1974-84, 2004
Pittler, S. & Baehr, W., Proc. Natl. Acad. Sci. USA, 88:8322-6, 1991
Ranclo, T. & Zhou, L., Proc. Natl. Acad. Sci. USA, 97: 5363-8, 2000
Rootman, D, et al., Invest. Opthalmol. Vis. Sci., 29:1397-401, 1988
Sakamoto, T. et al., Adv. Drug Deliv. Rev., 52:93-102, 2001
Santi, P. et al., Int. J. Pharm., 266:69-75, 2003
Sanyal, S. & Bal, A., Z Anat Entwicklungsgesch, 142:219-38, 1973
Sarraf, D. et al., Am. J. Opthalmol., 115:748-54, 1993
Silverman, M. & Hughes, S., Curr. Eye Res., 9:183-91, 1990
Sorensen, C. et al., J. Mol. Med., 83:39-49, 2005
Tagalakis, A. et al., J. Biol. Chem., 276:13226-30, 2001
Taubes, G., Science, 298:2116-2120, 1998
Voigt, M. et al., Biochem. Biophys. Res. Commun., 295:336-41, 2002
Wahlin, K. et al., Exp. Eye Res., 73:693-701, 2001
Wu, X. et al., J. Biomed. Sci., 8:439-45, 2001
Yamamoto, T. et al., Yeast, 8:935-48, 1992

### EQUIVALENTS

While the invention has been described in connection with the specific embodiments thereof, it will be understood that it is capable of further modification. Furthermore, this application is intended to cover any variations, uses, or adaptations of the invention, including such departures from the present disclosure as come within known or customary practice in the art to which the invention pertains, and as fall within the scope of the appended claims.

All references cited above are incorporated herein by reference.

We claim:

1. A method for enhancing the in vivo delivery of a nucleic acid into retinal cells of a mammalian eye, comprising the steps of:
(a) transiently elongating Müller cells of a mammal retina by a step of iontophoresis; and
(b) administering a composition comprising the nucleic acid to the mammalian eye,
wherein the step of iontophoresis transiently elongates the Müller cells of the mammal retina and enhances the in vivo delivery of the nucleic acid into the retinal cells of the mammalian eye.

2. The method of claim 1, wherein the nucleic acid is a therapeutic agent or a diagnostic agent.

3. The method of claim 1, wherein the nucleic acid is selected from the group consisting of: a deoxyribonucleic acid (“DNA”), a ribonucleic acid (“RNA”), and a chimeric nucleic acid comprising both DNA and RNA bases.

4. The method of claim 3, wherein the nucleic acid is selected from the group consisting of: an oligonucleotide DNA, an anti-sense DNA, a plasmid DNA, a component of a plasmid DNA, a vector, an expression cassette, a chimeric DNA sequence, a chromosomal DNA, a stabilized DNA, an aptamer, a stabilized aptamer, an oligonucleotide RNA, a transfer RNA (tRNA), a short interfering RNA (siRNA), a small nuclear RNA (snRNA), a ribosomal RNA (rRNA), an mRNA (messenger RNA), a micro RNA (miRNA), a short hair-pin RNA (shRNA), an antisense RNA, a ribozyme, a stabilized RNA sequence, a chimeric RNA sequence, a chimeric DNA/RNA oligonucleotide, an aptameric oligonucleotide, and a derivative of any of these nucleic acid.

5. The method of claim 4, wherein said nucleic acid is an oligonucleotide DNA or an oligonucleotide RNA, optionally with phosphorothioates linkages.

6. The method of claim 1, wherein the nucleic acid is selected from the group of: a single-stranded nucleic acid, a double-stranded nucleic acid, a triple-stranded nucleic acid, and a quadruple-stranded nucleic acid.

7. The method of claim 6, wherein the nucleic acid is in a linear or cyclic form.

8. The method of claim 7, wherein said nucleic acid is a single stranded oligonucleotide DNA (ssODN) or a single stranded oligonucleotide RNA (ssORN).

9. The method of claim 1, wherein the nucleic acid composition is administered either by a topical instillation or by an injection into the mammalian eye.

10. The method of claim 9, wherein the topical instillation is in the form selected from the group consisting of: a liquid solution, a paste, and a hydrogel.

11. The method of claim 10, wherein the topical instillation is embedded in a foam matrix.

12. The method of claim 10, wherein the topical instillation is supported in a reservoir.

13. The method of claim 9, wherein the injection is selected from the group consisting of: an intracameral injection, an intracorneal injection, a subconjonctival injection, a subtenon injection, a subretinal injection, an intravitreal injection, and an injection into the anterior chamber.

14. The method of claim 1, wherein the step of iontophoresis is an ocular or a transpalpebral iontophoresis.

15. The method of claim 14, wherein the transpalpebral iontophoresis is an anionic or a cationic iontophoresis performed with a current of about 1-5 mA for about 1-7 minutes.

16. The method of claim 15, wherein the transpalpebral iontophoresis is an anionic or a cationic iontophoresis performed with a current of about 1-3 mA for about 3-6 minutes.

17. The method of claim 16, wherein the transpalpebral iontophoresis is a cationic iontophoresis performed with a current of about 2 mA for about 5 minutes.

18. The method of claim 14, wherein the step of iontophoresis is carried out prior to, during, or after the step of administering said nucleic acid composition.


SEQUENCE LISTING (13 sequences)

SEQ ID NO: 1 - DNA, 68 bp, Artificial Sequence
  Description of Combined DNA/RNA Molecule DNA/2′OMeRNA derived from the murine gene encoding the cGMP-phosphodiesterase beta-subunit
ccttccaacctacgtagcagaaagtttttacuuucugcuacgtagguuggaagggcgcgttttcgcgc

SEQ ID NO: 2 - DNA, 25 bp, Artificial Sequence
  Description of Artificial Sequence DNA derived from the murine or human gene encoding the transcription factor HIF1alpha
ccatgtgaccattaggaaatgagag

SEQ ID NO: 3 - DNA, 24 bp, Artificial Sequence
  Description of Artificial SequenceDNA derived from the human gene encoding the opsin
gctttctttgccaagagcgccgca

SEQ ID NO: 4 - DNA, 22 bp, Artificial Sequence
  Description of Artificial Sequence DNA derived from the human gene encoding the RP1
aagaaaaaatctagacaagcaa

SEQ ID NO: 5 - DNA, 25 bp, Artificial Sequence
  Description of Artificial Sequence DNA derived from the murine gene encoding the opsin
gctttctttgctgagagctcttcca

SEQ ID NO: 6 - DNA, 22 bp, Artificial Sequence
  Description of Artificial Sequence DNA derived from the murine gene encoding the RP1
aagacttctgagtaacaatcaa

SEQ ID NO: 7 - DNA, 25 bp, Artificial Sequence
  Description of Artificial Sequence DNA derived from the murine or human gene encoding the transcription factor HIF1alpha
ccatgtgaccatgaggaaatgagag

SEQ ID NO: 8 - DNA, 24 bp, Artificial Sequence
  Description of Artificial Sequence DNA derived from the human gene encoding the opsin
gctttctttgccgagagcgccgca

SEQ ID NO: 9 - DNA, 15 bp, Artificial Sequence
  Description of Artificial Sequence DNA derived from the human gene encoding the RP1
aagaaaaaatcttga

SEQ ID NO: 10 - DNA, 25 bp, Artificial Sequence
  Description of Artificial Sequence DNA derived from the human gene encoding the RP1
gctttctttgctaagagctcttcca

SEQ ID NO: 11 - DNA, 68 bp, Artificial Sequence
  Description of Combined DNA/RNA Molecule DNA/2′OMeRNA derived from the murine gene encoding the cGMP-phosphodiesterase beta-subunit
ctaccaaatccatgggatttccatcagttauuucuguccatcagguaggagugggctcgcgtgcgttc

SEQ ID NO: 12 - DNA, 21 bp, Artificial Sequence
  Description of Artificial Sequence oligonucleotide sens (primer) derived from the murine cDNA encoding the cGMP-phosphodiesterase beta-subunit
ggccgggaaattgtcttctac

SEQ ID NO: 13 - DNA, 21 bp, Artificial Sequence
  Description of Artificial Sequence oligonucleotide reverse(primer) derived from the murine cDNA encoding the cGMP-phosphodiesterase beta-subunit
ccccaggaactgtgtcagaga